(19) United States
(12) Patent Application Publication
(10) Pub. No.: US 2007/0148715 A1
(43) Pub. Date: Jun. 28, 2007

(54) Method for detecting transient ligand interactions

(76) Inventor: Atanas Illiev Lalev, 225 London RD (CA)

Correspondence Address: BERESKIN AND PARR, 40 KING STREET WEST, BOX 401, TORONTO, ON, M5H 3Y2, CA

(21) Appl. No.: 10/568,409

(22) PCT Filed: Jul. 30, 2004
(86) PCT No.: PCT/CA04/01448

§ 371 Date: Sep. 26, 2006

Related U.S. Application Data
(60) Provisional application No. 60/494,811, filed on Aug. 14, 2003.
(60) Provisional application No. 60/545,493, filed on Feb. 19, 2004.
(60) Provisional application No. 60/566,396, filed on Apr. 30, 2004.

(30) Foreign Application Priority Data
Apr. 22, 2004 (CA) 2463301

(51) Int. Cl. G01N 33/554; G01N 33/569

(52) U.S. Cl. 435/7.32

(57) ABSTRACT

The invention relates to methods and kits for separating, and optionally analyzing, substoichiometrically interacting ligands from a high abundance ligand of interest in an affinity matrix system. The methods comprising immobilizing a complex of a first and second ligand to an affinity matrix, wherein the second ligand is bound to the matrix, and subsequently eluting the first ligand from the matrix. In addition, the methods and kits can be used for drug discovery.

### FIELD OF THE INVENTION

The invention relates to methods for separating and/or analyzing ligands in an affinity matrix system. In particular, the invention relates to separating substoichiometrically interacting ligands from high abundance ligand of interest in an affinity matrix system.

### BACKGROUND OF THE INVENTION

An important scientific pursuit in the post-genomic era is to determine the components of multiprotein complexes and to identify the protein-protein interactions in a proteome. The proteome consists of stable protein complexes and single (free) proteins but virtually every cellular process is mediated by and/or consists in transient protein interactions, e.g. protein-protein interactions, DNA-protein interactions or RNA-protein interactions. The detection and analysis of the transient interactions is a major challenge in biology and proteomics.

One method for protein purification and detection of protein interactions is creating fusion proteins by using recombinant DNA techniques. For example, the fusion protein can contain a protein of interest and an affinity tag capable of binding selectively to an affinity matrix. After expressing the fusion protein in exogenous or endogenous organism, it is immobilized on an affinity matrix and the unbound substances are removed. Subsequently, the fusion protein is released from the affinity matrix. The method is used in order to purify the affinity tagged protein or, more recently, the affinity tagged protein and the proteins that associate with it. The advent of sensitive mass spectrometric methods for protein identification and improved affinity purification methods made possible the direct identification of protein complexes on a proteome-wide scale (Ho Y. et al Nature 2002, Gavin, A. C. et al. Nature 2002). A review by R. Aebersold and M. Mann, Nature, March 2003, Vol. 422, “Mass spectrometry-based proteomics” describes in more detail the state of the field.

More than 300 stable complexes have been identified in the model organism Saccharomyces cerevisiae and extensive proteome maps have been built. However, it became clear that the number of transient protein-protein interactions detected by affinity purification coupled with mass spectrometry is smaller (often by an order of magnitude) compared to the number of transient protein-protein interactions detected by genetic based methods and library based methods. This is due to the fact that when the fusion protein forms transient complexes with other proteins, the latter are isolated in substoichiometric amounts and most often are not detected.

When the fusion protein and the interacting proteins are isolated via an affinity tag, the bulk of the fusion protein is immobilized on the affinity matrix, whereas only that fraction of the interacting proteins which has been bound to the fusion protein in vivo is immobilized. When the fusion protein is released from the affinity matrix, the ratio between the proteins in the eluate remains the same and, as a result, the highly abundant fusion protein suppresses the identification and analysis of the substoichiometrically interacting proteins. The detection of transient protein-protein interactions (e.g. interactions between stable protein complexes or between two single proteins or between a stable protein complex and a single protein) is still very difficult and in most cases impossible. Examples of proteins involved in transient protein-protein interactions include protein substrates, modifying enzymes such as protein kinases, signal transducers, DNA polymerase, replication factors, RNA polymerase, and transcription factors.

The following example with RNA polymerase II and the transcription factors illustrates the difficulty of detecting transient protein-protein interactions and the general problem of detecting any transient protein interaction by using any affinity tagged protein from any organism. Eukaryotic RNA polymerase II is a permanent complex of twelve subunits and it forms transient complexes with different transcription factors. When a SpA-tagged subunit of RNA Polymerase II core complex from Saccharomyces cerevisiae is expressed under physiological conditions, it associates permanently with other subunits of the core complex and, as a part of the core complex, it interacts directly or indirectly with a variety of transcription factors by forming transient (weak) complexes. After preparation of a protein lysate and immobilization of the SpA-tagged subunit on IgG-beads, the other subunits of the core complex are present in approximately equal molar amounts but the transcription factors are present in substoichiometric amounts.

Several factors contribute to the unequal stoichiometries including: (a) during the preparation of the cellular lysate and subsequent purification steps, transient complexes containing the RNA Polymerase II core complex dissociate in different ways and each core complex remains associated with a different set of transcription factors, (b) during the synthesis of mRNA, the RNA Polymerase II core complex associates with different proteins at different stages of the transcription, e.g. initiating factors, mediator complex, elongation factors, termination factors, (c) during the transcription of different genes, RNA Polymerase II core complex associates with different sets of gene specific transcription factors, and (d) a certain amount of the RNA Polymerase II core enzyme is not involved in transcription and is not associated with other proteins.

As a result, the amounts of the subunits of the core complex on the affinity matrix are disproportionably higher than the amounts of the interacting proteins. The isolation of the complexes by releasing the SpA-tagged subunit from the IgG-sepharose matrix and their analysis by mass spectrometry leads to identification of the subunits of the core complex, subunits of the general transcription factor TFIIF and, in some cases, Spt5, Taf10, Ceg1, Ess1, Kin28 and Srb6. However, more than 50 proteins that interact physically with the RNA polymerase II core enzyme are reported in the literature (Myer V. E. and Young R. A., J. Biol. Chem 1998).

Even when the isolated RNA Polymerase II complexes are fractionated by electrophoresis, some of the twelve subunits of the core complex (most often Rpb1 and Rpb2) and their different modification forms and degradation products are present in large amounts in many bands and they suppress the identification and/or analysis of low abundance proteins by mass spectrometry. In this case, the mass spectrum contains very large peaks, resulting from the high abundance proteins (i.e. subunits of Polymerase II core complex), and small peaks, resulting from the low abundance interacting proteins (i.e. transcription factors). Most often, the small peaks are not even detected and the analysis of the spectrum leads to identification of only the major protein(s). This is due to the following problems in mass spectrometry:
(i) The resolution problem in mass spectrometry instruments. If a large peak and small peak are separated by only a few mass units (i.e. their molecular weight differs by only 3-4 Da), the detection of the latter is impossible.
(ii) The dynamic range problem in mass spectrometry (high background noise problem). The signals from the abundant peptides suppress the signal from the minor proteins, even if they are separated by more than 3-4 mu. The background of the mass spectrum is due to the presence of large numbers of ions with low intensity. They are mostly multiple-charge ions and fragment ions from the major peptides and they are not detected as signals but as a background noise in the mass spectrum. In addition, adducts that are bound to the abundant peptides increase the background (chemical noise).
(iii) Ionization of the MALDI-TOF samples (co-crystallized peptides and a UV-matrix) is most optimal when the molar ratio between the matrix and the peptides is between 2,000:1 and 20,000:1. Hence, it is impossible to achieve a good ionization, and consequentially a good spectrum, for both high abundance and low abundance peptides, when the ratio between their amounts is 100:1 or 1000:1 or higher.

The example demonstrates the problems that are encountered when transient interactions are investigated by affinity purification coupled with mass spectrometry. The problem is even more severe in gel-less protein identification approaches (e.g. LS/MS/MS) because of the absence of initial separation step (PAGE) of the isolated proteins.

Besides the dynamic range problem in mass spectrometry, other problems arise when low abundance proteins or other substances are analyzed together with high abundance ones: (a) cross-reactivity problem in immunoassays, (b) overloaded chromatography columns or overloaded protein gels, and as a result, poor resolution, (c) ambiguous results in enzymatic assays.

The present invention solves these problems in an affinity matrix system by separating the substoichiometrically interacting proteins and/or other biomolecules from the high abundance protein of interest.

### SUMMARY OF THE INVENTION

The present invention relates to methods of separating, and optionally analyzing, ligands that form transient complexes with other ligands in vivo.

In one example, the invention relates to methods of separating a first ligand from a second ligand in an affinity matrix system, wherein the first ligand associates in vivo with the second ligand and wherein the first ligand and the second ligand associate with each other by electrostatic forces, comprising the steps:
(a) obtaining a sample containing biological complexes that include the first ligand and the second ligand;
(b) immobilizing the second ligand on an affinity matrix;
(c) removing unbound substances from the affinity matrix;
(d) separating the first ligand from the immobilized second ligand, which remains bound to the affinity matrix during the separation, by decreasing the electrostatic forces between the first ligand and the second ligand; and
(e) optionally, analyzing the separated first ligand.

In another example, the invention relates to methods of separating a first ligand from a second ligand in an affinity matrix system, wherein the first ligand associates in vivo with the second ligand, wherein the first ligand and the second ligand associate with each other by electrostatic forces, and wherein the second ligand is a recombinant fusion protein containing at least one affinity tag, comprising the steps:
(a) introducing into a cell or organism a recombinant nucleic acid molecule encoding a fusion protein comprising the second ligand fused to at least one affinity tag that can selectively bind to an affinity matrix;
(b) expressing the fusion protein;
(c) obtaining a sample containing biological complexes that include the first ligand and the fusion protein;
(d) immobilizing the fusion protein on the affinity matrix via the affinity tag;
(e) removing unbound substances from the affinity matrix;
(f) separating the first ligand from the immobilized fusion protein, which remains bound to the affinity matrix during the separation, by decreasing the electrostatic forces between the first ligand and the fusion protein; and
(g) optionally, analyzing the separated first ligand.

In a further example, the invention relates to methods of separating a first ligand from a second ligand in an affinity matrix system, wherein the first ligand associates in vivo with the second ligand, wherein the first ligand and the second ligand associate with each other by electrostatic forces, and wherein the second ligand is a recombinant fusion protein containing at least two different affinity tags, comprising the steps:
(a) introducing into a cell or organism a recombinant nucleic acid molecule encoding a fusion protein comprising the second ligand fused to at least two different affinity tags that can selectively bind to different affinity matrixes;
(b) expressing the fusion protein;
(c) obtaining a sample containing biological complexes that include the first ligand and the fusion protein;
(d) immobilizing the fusion protein on a first affinity matrix via a first affinity tag;
(e) removing unbound substances from the first affinity matrix;
(f) separating the fusion protein from the first affinity matrix;
(g) immobilizing the fusion protein on a second affinity matrix via a second affinity tag, which is different than the first affinity tag;
(h) removing unbound substances from the second affinity matrix;
(i) separating the first ligand from the immobilized fusion protein, which remains bound to the second affinity matrix during the separation, by decreasing the electrostatic forces between the first ligand and the fusion protein; and
(j) optionally, analyzing the separated first ligand.

In another example, the invention relates to methods of separating a first ligand from a second ligand in an affinity matrix system, wherein the first ligand associates in vivo with the second ligand, wherein the first ligand and the second ligand associate with each other by electrostatic forces, and wherein the second ligand is a protein complex containing two or more subunits of which are fused to different affinity tags, comprising the steps:
(a) introducing into a cell or organism recombinant nucleic acids molecules encoding fusion proteins comprising the two or more subunits of which are fused to different affinity tags that can selectively bind to different affinity matrixes;
(b) expressing the fusion proteins;
(c) obtaining a sample containing biological complexes that include the first ligand and the fusion proteins;
(d) immobilizing the fusion proteins on a first affinity matrix via a first affinity tag;
(e) removing unbound substances from the first affinity matrix;
(f) separating the fusion proteins from the first affinity matrix;
(g) immobilizing the fusion proteins on a second affinity matrix via second affinity tag, which is different than the first affinity tag;
(h) removing unbound substances from the second affinity matrix;
(i) separating the first ligand from the immobilized fusion proteins, which remain bound to the second affinity matrix during the separation, by decreasing the electrostatic forces between the first ligand and the fusion proteins; and
(j) optionally, analyzing the separated first ligand.

The method of the invention is suitable for detecting transient protein-protein interactions, and other transient interactions that involve proteins, including but not limited to DNA-protein interactions, RNA-protein interactions, carbohydrate-protein interactions, and lipid-protein interactions.

Another aspect of the invention is a method for drug discovery. A chemical or biomolecule can be identified as a drug or a pre-drug by its ability to selectively affect a particular protein interaction that is associated with a disease.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 shows exploded views of a chromatography column packed with ligand coated solid support: 1—chromatography column packed with affinity beads, 2—an expanded view of several individual beads, 3—an expanded view of an individual bead of affinity matrix, 4—solid support (e.g. agarose or sepharose), 5—ligand (e.g. IgG or glutathione) that binds selectively to affinity tag, 6—linker between the solid support and the ligand.

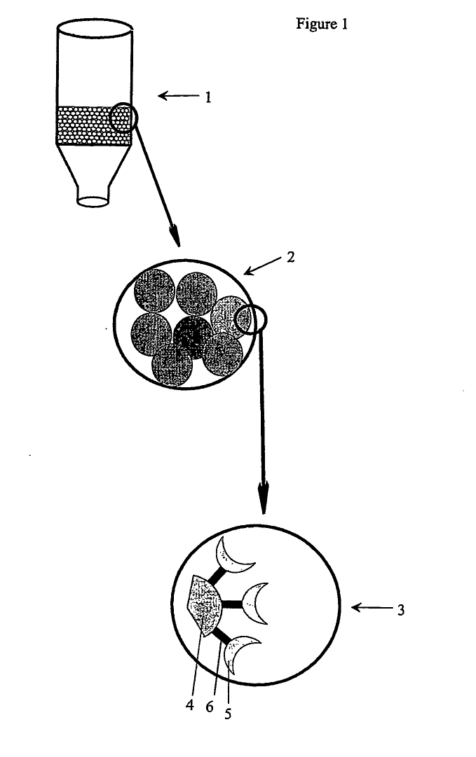

FIG. 2 illustrates the two essential steps (bold vertical arrows) of the invention Affinity Purification with Switching the Ligand (APSL). Legend: 7—linker between the protein of interest and the affinity tag, 8—affinity tag that is fused to a protein of interest; 9—protein of interest or subunit of a permanent protein complex that is affinity tagged (i.e. fusion protein); 10, 11, 12, 13, 14, 15—proteins that interact directly with the fusion protein or protein complex; 19—protein that interacts indirectly with the fusion protein or protein complex; 16—the fusion protein or a protein complex and the interacting proteins are immobilized on the affinity matrix; 17—the fusion protein or protein complex remains immobilized on the affinity matrix after the elution; 18—the interacting proteins are separated into the liquid phase after the elution (the eluate is indicated with a dotted line); 20—Step One: preparation of cellular lysate or other biological fluid from an organism or cell line containing a heterologous nucleic acid expressing an affinity tagged protein and immobilizing the affinity tagged protein on the affinity matrix by the tag; removing the proteins and other substances that are not bound directly or indirectly to the fusion protein; 21—Step Two: separating the interacting proteins from the immobilized fusion protein by elution with an agent that does not disrupt the binding between the affinity matrix and the immobilized fusion protein or subunit of a permanent protein complex; only proteins and other substances that bind directly or indirectly to the immobilized protein are eluted. The bonds between some of the affinity tags and their respective ligands are mainly hydrophobic and it is therefore reasonable to increase the ionic strength of the medium in Step Two in order to weaken the electrostatic attractions by Debye-Huckel screening.

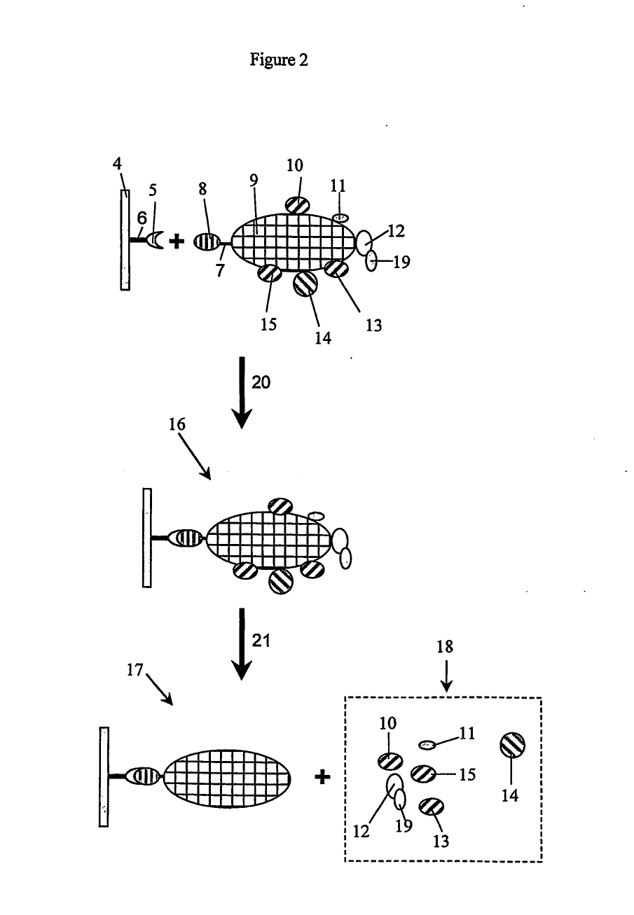

FIG. 3 is a gel image of high salt eluate from immobilized RNA Polymerase II from Saccharomyces cerevisiae. The fusion protein contains Rpb1 and a SpA tag containing four IgG binding domains. The digits and arrows at the right side of the gel indicate the identified protein. Legend: 31—Spt6, 32—Rgr1, 33—Spt6+Spt5, 34—Tfg1+Spt5, 35—Set3, 36—Cet1+Med1, 37—Pob3, 38—Npl3, 39—Tfg2+Iws1, 40—Cdc73, 41—TFIIS, 42—Ess1.

FIG. 4 is a gel image of high salt eluate from immobilized RNA Polymerase II from Saccharomyces cerevisiae. The fusion protein contains Rpb1 and a SpA tag containing two IgG binding domains. Legend: 51—Spt5+Spt6, 52—Spt5+Spt6, 53—Spt5+Spt6, 54—Ctr9+Spt5+Spt6, 55—Spt5+Ydl145 (Cop1)+Spt6, 56—Tfg1+Spt5+Spt6+Spt16, 57—Spt5+Tfg1+Sec21, 58—Spt5+Tfg1, 59—Set2+Spt5+Rtf1+Ydl145 (Cop1), 60—Cet1, 61—Tfg2+Iws1, 62—Ceg1, 63—Cdc73, 64—TFIIS, 65—Rtt103, 66—Tfg3, 67—Yhr121w, 68—Ess1.

FIG. 5 is a gel image which identifies proteins that bind nonspecifically to IgG beads and/or to other proteins (contaminants). Legend: 70—Acc1+Fas1+Fas2, 71—Lys2+Ydr098, 72—Mdn1+Tra1, 73—Sec27+Ade3, 74—Prt1+Lys2, 75—Sse1+Lsg1, 76—Ssb1+Ssa2+Pab1+Ded1, 77—Hsp60+Dhh1, 78—Gcd11+Ded1+Nop12+Nog2, 79—Tef2+Phr1, 80—Tef2+L3, 81—Nop3+Tef2, 82—Yor060+Ydj1, 83—Stm1+Rpl4, 84—Tif34, 85—Rpp0+Rpl5, 86—Tef2+Nop1, 87—Rps1+Rpl2, 88—Rps3+Rpl8, 89—Rps2, 90—Rps5, 91—Rps8+Rpl9, 92—Rpl10, 93—Rps20, 94—Rpl20, 95—Rps14, 96—Rps17, 97—Rpl27+Rps24.

FIG. 6 is a gel image of high salt eluates from immobilized RNA Polymerase II complexes from Saccharomyces cerevisiae expressing a Rpb1-Sp-A tagged fusion protein. Legend: 120—Spt6, 121—Spt6, 122—Spt5+Cyr1+Clu1, 123—Tfg1+Nam7+Spt16, 124—Fcp1+Cdc48+Sec21, 125—Taf5+Top2, 126—Cet1+Pcf1+Prp6, 127—Pob3, 128—Rtt103+Ptc3+Has1, 129—Tfg2+Iws1+Pap1, 130—Tfg2+Iwr1; 131—Rai1, 132—Sua7, 133—TFIIS, 134—Ypl253, 135—Ssn3+Mrt4, 136—Tfg3+Ptc1, 137—Med7, 138—Yhr121w, 139—Mbf1+Taf9+Yor379c, 140—Ybr262c+Ybr174c.

FIG. 7 is a gel image of high salt eluate from immobilized RNA. Polymerase II complexes from Saccharomyces cerevisiae expressing a Rpb3-Sp-A tagged fusion protein. Legend: 150—Spt6, 151—Spt5+Ctr9+New1, 152—Spt16+Tor2, 153—Tfg1+Hpr5+Nam7, 154—Fcp1+Cdc48+Nam7, 155—Fcp1+Rad3, 156—Fcp1+Taf5+Pbp1, 157—Cet1+Cyr1, 158—Pob3, 159—Rtt103+Ptc3, 160—TFIIH+Rad23+Drs1, 161—Tfg2+Iws1, 162—Rai1+Pob3+Rgr1+Ygr086c, 163—Fun11+Sub1, 164—TFIIB, 165—TFIIS+Yol045, 166—Tfg3, 167—Din7, 168—Yhr121w, 169—Nhp2, 170—Mbf1.

FIG. 8 is a gel image of high salt eluate from immobilized RNA Polymerase II complexes from Saccharomyces cerevisiae expressing a Rpb9-Sp-A tagged fusion protein. Legend: 180—Spt6+Stt4+Ydl145 (Cop1), 181—Spt5+New1+Tof1, 182—Spt5+Rad3, 183—Tfg1+Cdc48+Rgr1+Nam7, 184—Fcp1+Cdc48+Rad1+Sec21, 185—Fcp1+Sup35+Rap1, 186—Cet1+Rap1+Cdc6, 187—Tfg2+Iws1, 188—Ceg1+Kin1+Prp8, 189—Ceg1+Rgm1, 190—Ygr086c+Ydr065w, 191—TFIIB, 192—TFIIS+Tho1+Bim1, 193—Tfg3+Tfa2 (TFIIE), 194—Set3+Tfb2 (TFIIH)+Yol022c. Cdc6, Prp2, Prp16, Rif1 and Cft1 were also detected.

FIG. 9 is a gel image of high salt eluate from immobilized TFIIF from Saccharomyces cerevisiae. The carboxy-terminus of the chromosomal copy of the gene encoding Tgf1 is fused in-frame with a TAP-tag. Legend: 201—Rpb1, 202—Rpb1+Rpb2, 203—Fcp1+Rpb2+Rpb1, 204—Rpb1+Rpb2+Fcp1 , 205—Glo3, 206—Rpb3, 207—TFIIS, 208—Mpd2, 209—Rpb4, 210—Yhr121w+Rpb5, 211—Rpb6, 212—Rpb7, 213—Mbf1+Rpb8, 214—Rpb9.

FIG. 10 is a gel image of high salt eluate from immobilized RNA Polymerase II from Saccharomyces cerevisiae. The carboxy-terminus of the chromosomal copy of the gene encoding Rpb1 is fused in-frame with a TAP-tag. Legend: 221—Rpb1, 222—Spt6, 223—Spt6, 224—Rpb2+Ctr9, 225—Ctr9+Spt6, 226—Tfg1+Prp22+Spt16+Spt6, 227—Tfg1+Fcp1+Spt16+Nip1, 228—Fcp1+Set2+Rtf1+Ygl244, 229—Tfg1+Fcp1+Ygr054+Leo1+Med1, 230—Pob3, 231—Paf1+Glo3, 232—Iws1+Tfg2, 233—TFg2, 234—Sua7(TFIIB homolog), 235—TFIIS, 236—Mpd2, 237—Tfg3, 238—Ess1, 239—Mbf1.

FIG. 11 is a gel image of high salt eluate from immobilized RNA Polymerase II from Saccharomyces cerevisiae (right lane) and the subsequentially released core complex (left lane). The carboxy-terminus of the chromosomal copy of the gene encoding Rpb1 is fused in-frame with a TAP-tag. Legend: 271—Spt6, 272—Ydl145 (Cop1), 273—Ctr9+Spt5, 274—Tfg1+Spt16+Lte1, 275—Tfg1+Spt5+Spt16+Fcp1, 276—Set2, 277—Fcp1+Leo1, 278—Pob3, 279—Paf1, 280—Paf1+Rtt103, 281—Tfg2+Iws1, 282—Tfg2, 283—Cdc73+Ceg1, 284—Dst1, 285—Rtt103, 286—Tfg3, 287—Ess1, 288—Rpb1, 289—Rpb2, 290—Tfg1, 291—Tfg2, 292—Rpb3, 293—Tfg3, 294—Rpb4, 295—Rpb5, 296—Rpb6, 297—Rpb7, 298—Rpb8.

FIG. 12 is a gel image of the high salt eluate from immobilized Rpb3 from Saccharomyces cerevisiae. The carboxy-terminus of the chromosome copy of the gene encoding Rpb3 is fused in-frame with a TAP-tag. The interacting proteins are indicated. Trace amounts of Rpb1, Rpb2 and Rpb3 are also indicated.

FIG. 13 shows a comparison between the method of the invention and other methods for detecting protein-protein interactions that utilize affinity tagged proteins. Legend: 301—the left panel illustrates GST pull down, 302—the middle panel illustrates purification via an affinity tag, 303—the right panel illustrates the method of the invention. 310—exogenous organism (e.g. Escherichia coli), 311—endogenous organism, 312—affinity tagged protein, 313—the affinity tagged protein and the interacting proteins are immobilized on solid phase, 314—unbound proteins and/or other substances (solid black) are removed, 315—isolating the interacting proteins—only in the case of the invention they are separated from the affinity tagged protein (the eluate does not contain affinity tagged protein), 316—eluate obtained by GST pull down; 317—eluate obtained by purification via an affinity tag; 18—eluate obtained by the method of the invention.

FIG. 14 illustrates the necessity to separate substoichiometrically interacting proteins from the high abundance fusion protein. The bulk of the fusion protein is immobilized via an affinity tag whereas only the fraction of interacting proteins that has been bound to the fusion protein in vivo is immobilized. 330—transient complexes are immobilized on an affinity matrix via an affinity tag, 331—purification via an affinity tag, 332—purification by the method of the invention, 317—eluate obtained by purification via an affinity tag, 18—eluate obtained by the method of the invention.

FIG. 15 illustrates the formation and disintegration of a transient protein complex between two stable (permanent) protein complexes (first and second vertical arrows respectively). Legend: 351—stable protein complex with 4 subunits, 352—stable protein complex with 6 subunits, 353—transient protein complex between the two stable complexes, 354—subunit of a stable complex that binds indirectly to another stable complex, 355—formation of a transient protein complex, 356—disintegration of the complex.

FIG. 16 is a model for transient protein-protein interactions according to which formation and disintegration of transient protein complexes is due to alternation of electrostatic attraction and electrostatic repulsion between the electrostatically charged amino acids. Legend: 370—first interacting protein, 371—second interacting protein, 372—positively charged amino acids, 373—negatively charged amino acids, 374—electrostatic attraction, 375—two interacting proteins form a complex, 376—change of position or orientation of the two interacting proteins relative to one another, 377—electrostatic repulsion, 378—transient complex disintegrates.

FIG. 17 illustrates that when a mutation changes the electrostatic properties of the protein, the transient interaction cannot occur properly. Legend for the upper panel: 380—mutation leading to disappearance of positively charged amino acid, 381—electrostatic attraction is prevented. Legend for the lower panel: 382—mutation leading to disappearance of different positively charged amino acid, 383—electrostatic repulsion is prevented and the interacting proteins remain stuck together.

FIG. 18 shows a method for drug design and schematic structure of a chemical or a biomolecule that binds to a mutant protein and restores (at least partially) its electrostatic properties. As a result, the protein is capable of forming a protein complex. Legend: 390—drug treatment, 391—a drug, 392—an exploded view of the drug, 393—part that mimics and/or restores the original charge, 394—linker (optional), 395—part that binds selectively to the mutant protein.

FIG. 19 illustrates the selective effect of a drug on a particular protein-protein interaction. Legend: 430—left panel: the immobilized protein of interest is treated with nonspecific elution agent and all interacting proteins are separated, 431—middle panel: the immobilized protein of interest is treated with specific elution agent which causes separation of only one interacting protein complex, 432—right panel: the unrelated protein complex is treated with the same specific elution agent but there is no separation of the interacting proteins, 438—unrelated protein, 439—protein interacting with the unrelated protein, 449—another protein interacting with the unrelated protein, 440—elution with nonselective elution agent (e.g. increased salt concentration), 441—elution with selective agent (e.g. drug treatment), 18—eluate obtained by the method of the invention, i.e. proteins eluted by treatment with a nonselective elution agent, 445—proteins eluted by treatment with a selective agent; 446—the unrelated protein complex is not affected by the treatment with a selective agent and the interacting proteins are not eluted.

FIG. 20 illustrates elution by a gradient and collecting different fractions and analyzing each one (by immunodetection for example) for the presence of the affinity tag or the affinity tagged protein: 401—chromatography column; 402—nine subsequent fractions, small amount of each one are blotted to a membrane and analyzed; 411, 412, 413, 414, 415, 416—blots corresponding to fractions that do not contain affinity tagged protein, 417, 418, 419—blots corresponding to fractions that contain increasing amounts of the affinity tagged protein.

FIG. 21 illustrates the problems with analyzing fractions or gel bands containing proteins with different abundances by mass spectrometry. After the trypsin digest, the concentration range of the peptides remains approximately the same and it results in mass spectrum containing peaks with different intensity. Left panel: When the mass spectrum contains large and small peaks separated by only a few (1-5) mass units, the detection of the latter is impossible because of the dynamic range problem in mass spectrometry. Analyzing such spectrum leads to the identification of only the major protein(s) in the band. Right panel: High background noise problem in mass spectrometry—when a protein mixture with high dynamic range (ratio between the most abundant and the least abundant protein) is analyzed directly the small peptides are not detected because their intensities are lower than the background. Legend: 450—high abundance peptide; 451—different low abundance peptides; 452—background of the mass spectrum; 453—low abundance peptide is not detected because it is obscured by a high abundance peptide which mass is two mass units smaller; 454—the separation of the substoichiometrically interacting proteins from the high abundance protein of interest results in lower background (due to the absence of the high abundance peptides) and makes possible the detection of the low abundance peptides.

FIG. 22 shows the two subunits of the RNA Polymerase II core complex (Rpb1 and Rpb2) co-purified with Rtt103. Legend: 470—Rpb1, 471—Rpb2, 472—Rat1, 473—Rtt103, 474—Rai1.

### DETAILED DESCRIPTION OF THE INVENTION

The invention relates to methods for separating, and optionally analyzing, ligands in an affinity matrix system. In particular, the invention relates to separating, and optionally analyzing, substoichiometrically interacting ligands from the high abundance ligand of interest in an affinity matrix system.

In one example, the invention relates to methods of separating a first ligand from a second ligand in an affinity matrix system, wherein the first ligand associates in vivo with the second ligand and wherein the first ligand and the second ligand associate with each other by electrostatic forces, comprising the steps:
(a) obtaining a sample containing biological complexes that include the first ligand and the second ligand;
(b) immobilizing the second ligand on an affinity matrix;
(c) removing unbound substances from the affinity matrix;
(d) separating the first ligand from the immobilized second ligand, which remains bound to the affinity matrix during the separation, by decreasing the electrostatic forces between the first ligand and the second ligand; and
(e) optionally, analyzing the separated first ligand.

In another example, the invention relates to methods of separating a first ligand from a second ligand in an affinity matrix system, wherein the first ligand associates in vivo with the second ligand, wherein the first ligand and the second ligand associate with each other by electrostatic forces, and wherein the second ligand is a recombinant fusion protein containing at least one affinity tag, comprising the steps:
(a) introducing into a cell or organism a recombinant nucleic acid molecule encoding a fusion protein comprising the second ligand fused to at least one affinity tag that can selectively bind to an affinity matrix;
(b) expressing the fusion protein;
(c) obtaining a sample containing biological complexes that include the first ligand and the fusion protein;
(d) immobilizing the fusion protein on the affinity matrix via the affinity tag;
(e) removing unbound substances from the affinity matrix;
(f) separating the first ligand from the immobilized fusion protein, which remains bound to the affinity matrix during the separation, by decreasing the electrostatic forces between the first ligand and the fusion protein; and
(g) optionally, analyzing the separated first ligand.

One distinctive feature of the invention is that the second ligand remains immobilized on the affinity matrix (directly or indirectly), but the first ligand, which is the interacting molecule of interest, is separated into a liquid phase, which can be analyzed separately.

Unless otherwise indicated, the terms “high abundance”, “low abundance”, “high amount” and “low amount” designate the amounts of the ligands when they are immobilized on the affinity matrix but not their amounts in the organism or cell.

The definition of a “transient protein complex” as used herein is a complex with components that exist as both complexes and free proteins through electrostatic interactions.

The term “transient ligand complex” as used herein refers to a complex of components that exist as both complexes and free ligands through electrostatic interactions.

The term “interacting ligand” as used herein refers to the first ligand, which is the ligand that forms a transient ligand complex with the ligand of interest (second ligand) and it is not to be confused with the subunits of a permanent ligand complex.

The term “ligand of interest” as used herein refers to the second ligand and designates the ligand whose partners are sought.

The term “associated ligand” as used herein refers to ligands that form either stable or transient complexes with the ligand of interest (second ligand).

The term “ligand” as used herein refers to a molecule that can bind another molecule and includes, but is not limited to proteins, RNA, DNA, lipids and carbohydrates.

The term “affinity matrix system” as used herein means a system where a particular ligand is immobilized or coupled to a solid support so that when a sample comes into contact with immobilized ligand, only those molecules in the sample having specific binding affinity to the ligand are bound to the immobilized ligand. An affinity matrix system makes use of specific binding interactions between molecules.

The following terms, synonyms and abbreviations are used herein:

protein interaction=interaction involving protein (e.g. protein-protein, DNA-protein, RNA-protein, carbohydrate-protein, lipid-protein);

ligand interaction=interaction involving ligands (e.g. proteins, DNA, RNA, carbohydrates and/or lipids);

protein of interest=protein whose partners are sought;

ligand of interest=ligand whose partners are sought=second ligand;

weak protein interaction=transient protein interaction;

weak ligand interaction=transient ligand interaction;

stable protein complex=permanent protein complex;

stable ligand complex=permanent ligand complex;

high salt eluate=eluate that has been obtained by increasing the ionic strength of the medium surrounding the immobilized complexes;

SpA-tag=affinity tag consisting of one or more IgG binding domains genetically derived from Protein A from Staphylococcus aureus;

SpG-tag=affinity tag consisting of one or more IgG binding domains genetically derived from Streptococcal protein G (strain G148);

unbound substances=substances that are not immobilized directly or indirectly on the affinity matrix;

protein complex=biological complex containing one or more protein components;

ligand complex=biological complex containing one or more ligand components;

washing buffer=the buffer that is used to remove the unbound substances;

elution buffer=the buffer that is used to separate the interacting ligand (first ligand) from the immobilized ligand of interest (second ligand). In an example of the method of the invention, the protein of interest (second ligand) is a fusion protein and the elution buffer is washing buffer with an increased concentration of KCl or other salt and/or other substances.

pre-drug=drug candidate=chemical that is tested in order to determine whether it selectively affects a particular complex associated with a disease;

SDS-PAGE=polyacrylamide gel electrophoresis in the presence of sodium dodecyl sulphate;

M=molar (concentration);

mM=millimolar;

mcM=micromolar;

ml=milliliter;

mcl=microliter;

rpm=revolutions per minute (during centrifugation);

MBP=maltose binding peptide;

SBP=streptavidin binding peptide;

WB=washing buffer;

ORF=open reading frame;

contaminant protein=protein that binds nonspecifically to the protein of interest or to any protein or to the affinity matrix; and

MALDI-TOF MS=matrix assisted laser desorption ionization/time of flight mass spectrometry.

As stated above the term ligand complex means a biological complex containing one or more ligand components. The ligands can be proteins, nucleic acids, lipids or carbohydrates. The ligands can be modified (e.g. proteins can be glycoproteins or lipoproteins).

The term “biological complex” means an association of ligands in a biological sample. The ligands can include proteins, DNA, RNA, carbohydrates and/or lipids present in a biological sample.

The term “sample containing biological complexes” or “biological sample” as used herein means a cellular lysate, a cell lysate, a fraction of a cellular lysate or biological fluid that contains biological complexes. The sample can be from any animal, in particular humans, and may be biological fluids (such as blood, saliva or cerebrospinal fluid), tissue, hair or organs. In a preferred embodiment, the sample is a cell lysate. In one example, the cell lysate is obtained from a cell sample (e.g. culture of cells, tissue, or biological fluid) by using mechanical forces on the cell sample, such as grinding the frozen sample, glass beads beating, brief sonication or homogenization. In addition, lysis buffer can be used, such as 2×Lysis Buffer (200 mM KCl, 50 mM HEPES pH 7.4, 20% glycerol, 2% DMSO, 0.2% TritonX100, 2 mM PMSF, 2 mM benzamidine, 2×phosphatase inhibitors). It is advisable to keep the temperature between 0° C. and 10° C., and maintain the pH optimally close to a physiological pH.

The invention can be carried out with whole cells, cellular organelles, cellular lysate, or other biological fluids. In addition, the invention can be performed with fusion proteins and interacting ligands expressed in a cell free system. Cellular lysate can be fractionated in order to obtain a fraction that is enriched in the ligand of interest. When the organism is a multicellular organism, the immobilized fusion protein can originate from one tissue but the interacting ligands can originate from a different tissue (e.g. hormones). In this case, the biological fluid containing the interacting ligands can be applied to the fusion protein before or after the immobilization of the fusion protein. When the biological fluid containing the interacting ligands is applied after the immobilization of the fusion protein and removal of the unbound substances, the immobilized fusion protein can be associated with ligands from its original tissue or, the associated ligands can be removed before applying the other biological fluid originating from a different tissue. The fusion protein can be cross-linked to the affinity matrix in order to minimize the leakage.

The term “immobilizing” as used herein means the second ligand is immobilized to the affinity matrix either directly or indirectly. For example, if the second ligand is a glycoprotein, then it can be immobilized directly to an affinity matrix coated with lectin (e.g. lectin coated beads). In other examples, the second ligand can be indirectly immobilized to the affinity matrix. For example, the second ligand can be joined to an affinity tag, such as, but not limited to, SpA, SpG, GST, SBP, SBP, poly-His or Flag, which can selectively bind to the respective affinity matrix. In addition, the second ligand can be indirectly immobilized to the affinity matrix via antibodies specific for the second ligand. For example, if the anti-second ligand antibody is IgG type then the antibody can be immobilized to a Protein A Sepharose affinity matrix, and then the second ligand can be indirectly immobilized to the affinity matrix via the anti-second ligand specific antibody. In another example, the anti-second ligand antibody can be biotinylated, and then the biotinylated antibody can be immobilized to a streptavidin affinity matrix, and then the second ligand can be indirectly immobilized to the affinity matrix via the anti-second ligand specific antibody.

The term “antibody” as used herein is intended to include monoclonal antibodies and polyclonal antibodies, antibody fragments (e.g. Fab and F(ab′)₂, and single chain antibodies (scFV)), and chimeric antibodies, which also specifically react with a ligand, such as the second ligand in the method of the invention. Antibodies can be fragmented using conventional techniques and the fragments screened for utility. For example, F(ab′)₂ fragments can be generated by treating antibody with pepsin. The resulting F(ab′)₂ fragment can be treated to reduce disulfide bridges to produce Fab′ fragments. Papain digestion can lead to the formation of Fab fragments. Single chain antibodies combine the antigen-binding regions of an antibody on a single stably folded polypeptide chain. Single chain antibodies can be generated by recombinant technology.

Antibodies having specificity for a ligand may be prepared by conventional methods. A mammal, (e.g. a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g. the hybridoma technique originally developed by Kohler and Milstein (Nature 256:495-497 (1975)) as well as other techniques such as the human B-cell hybridoma technique (Kozbor et al., Immunol. Today 4:72 (1983)), the EBV-hybridoma technique to produce human monoclonal antibodies (Cole et al., Methods Enzymol, 121:140-67 (1986)), and screening of combinatorial antibody libraries (Huse et al., Science 246:1275 (1989)). Hybridoma cells can be screened immunochemically for production of antibodies specifically reactive with the peptide and the monoclonal antibodies can be isolated.

Specific antibodies, or antibody fragments, reactive against ligand components may also be generated by screening expression libraries encoding immunoglobulin genes, or portions thereof, expressed in bacteria with cell surface components. For example, complete Fab fragments, VH regions and FV regions can be expressed in bacteria using phage expression libraries (See for example Ward et al., Nature 341:544-546 (1989); Huse et al., Science 246:1275-1281 (1989); and McCafferty et al., Nature 348:552-554 (1990)). Alternatively, a SCID-hu mouse, for example the model developed by Genpharm, can be used to produce antibodies, or fragments thereof

“Joining the second ligand to an affinity tag” as used herein means the affinity tag is joined to the second ligand directly or indirectly. For example, the second ligand can be joined to the affinity tag indirectly via a linker, such as a peptide linker, a chemical linker, a nucleic acid linker or other biomolecule linker. In another example, the affinity tag can be joined to the second ligand directly. The fusion ligand, which is the second ligand joined directly or indirectly to an affinity tag, can be made using recombinant technology. Methods for purifying and manipulating recombinant proteins including fusion proteins are well known in the art. Necessary techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); “Current Protocols in Immunology” (J. E. Coligan et al., eds., 1991).

The term “removing unbound substances from the affinity matrix” as used herein means washing the affinity matrix with the immobilized second ligand bound in a manner that removes unbound substances from the affinity matrix while leaving the immobilized second ligand bound to the affinity matrix and the first ligand still associated with the second ligand. In one example, the wash buffer is 100 mM KCl, 10 mM HEPES pH 7.4, 5% glycerol, 1% DMSO, 0.1% TritonX100.

The term “separating the first ligand from the immobilized second ligand” as used herein means separating the first ligand from the immobilized second ligand in a manner that removes the first ligand from the second ligand while leaving the immobilized second ligand bound to the affinity matrix. The first ligand can be separated from the immobilized second ligand by disrupting the electrostatic forces between the first ligand and the immobilized second ligand. The electrostatic forces can be disrupted by using an elution buffer that increases the ionic strength of the medium. In one example, the ionic strength of the medium is increased using an elution buffer with an increased salt concentration, such as 0.3-0.5M potassium chloride (KCl) or 0.7M potassium acetate. One of skill in the art will appreciate that any salt that can increase the ionic strength of the medium can be used in the method of the invention. Examples of salts that may be used include, without being limiting, potassium chloride, sodium chloride, potassium acetate and sodium acetate.

Another variation of separating the first ligand from the immobilized second ligand in a manner that removes the first ligand from the second ligand while leaving the immobilized second ligand bound to the affinity matrix includes changing the pH of the system or enzymatic treatment that modifies the first ligand and/or second ligand.

A person skilled in the art will appreciate that any pH can be used to separate the first ligand from the immobilized second ligand, as long as it does not disrupt the bond between the affinity matrix and the immobilized second ligand. In one example, a pH range between 4 and 10 can be used to separate the first ligand from the immobilized second ligand. In another example, pH 4 can be used for separating the first ligand, which does not affect the IgG-SpA bond or IgG:SpG bond.

A person skilled in the art will also realize that different enzymatic treatments can be used. For example, non-specific phosphatases (alkaline and acid) can hydrolyze almost any phosphate ester, such as CIP, which is an alkaline phosphatase of calf intestines that catalyzes the non-specific hydrolysis of phosphate monoesters. Another example is serine/threonine protein phosphatases. These are relatively non-specific and can be used for treatment of a wide range of proteins. New England Biolabs offers YOP Protein Tyrosine Phosphatase (coded by Yop51 gene of Yersinia enterocolitica), which is a phospho tyrosine-specific protein phosphatase. By treating the immobilized complex with different specific phosphatases, one can determine which phosphorylated amino acid(s) take part in the formation of the salt bonds between the first and second ligand.

It is a variation of the method of the invention to add in the elution buffer substances that facilitate the separation of the interacting proteins from the protein of interest (e.g. 0.5M urea or 0.5M guanidinium chloride or SDS below 0.1% or TritonX100 below 1%), but that do not disrupt the bond between the affinity tag and the affinity matrix.

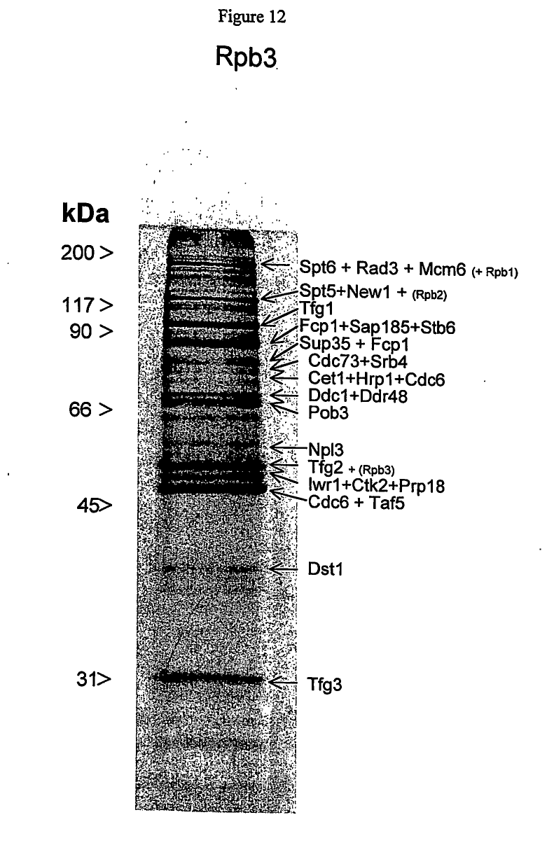

Elution can be performed by gradually increasing the concentration of the elution agent (e.g. salt gradient, pH gradient, enzyme concentration). A person skilled in the art will appreciate that as the concentration or strength of the elution agent increases, the possibility of the immobilized complex separating into the liquid phase increases. For example, FIG. 12 shows that during high salt elution trace amounts of Rpb1, Rpb2 and Rpb3 were separated into the liquid phase, which did not interfere with the successful identification of the interacting proteins. Different variations of elution agents can be assessed to eliminate or avoid leakage of the immobilized complex when separating the first ligand from the immobilized second ligand.

Another variant is to fractionate further the ligands in the eluate by chromatography (e.g. liquid chromatography) or other separating techniques. However, the fractionation may lead to unnecessary dilution and/or distribution of an interacting ligand in several fractions. In this case, all the fractions that do not contain the affinity tagged ligand, or contain only insignificant amount, can be recombined.

The elution agent might disrupt partially and nonselectively the binding between the affinity tag and the affinity matrix (especially if some affinity tags are partially denatured during the preparation of the cellular lysate) and, as a result, a certain amount of the immobilized fusion ligand can detach from the affinity matrix and co-elute with the interacting ligands. The problem can be solved in several ways:

(i) The affinity matrix can be pre-treated under the same conditions as the elution conditions in order to wash away any ligand or affinity material, such as IgG, Calmodulin, Amylose etc. that is not attached strongly enough to the solid support (e.g. agarose or sepharose);

(ii) The highest concentration of the elution agent that does not cause separation of the fusion ligand from the affinity matrix can be determined in a pilot experiment(s) using the same affinity tag fused to a standard (control) protein. Different fractions can be analyzed by immunodetection (with antibody against the affinity tag or against the standard protein) or other techniques in order to determine the presence or absence of the affinity tag and/or affinity tagged standard ligand. The elution conditions that do not lead to separation of the affinity tagged standard ligand from the affinity matrix should be used to perform the method of the invention with the ligand of interest;

(iii) The presence of an interacting ligand in a fraction that is eluted before the first fraction that contains the fusion ligand is sufficient to prove that the interacting ligand is isolated and/or detected by separating it from the immobilized fusion ligand; and

(iv) If the fusion ligand leaks together with the affinity matrix (e.g. IgG), this is a problem with the affinity matrix.

In one example, the affinity matrix is in the form of beads and can be packed into a column so that the removal of the unbound proteins and other substances can be achieved by washing the column. The affinity tag binds the affinity matrix by predominantly hydrophobic forces and the elution of the interacting ligands is carried out by increasing the ionic strength and/or the dielectric constant of the medium. The elution is performed under conditions that do not weaken the binding between the affinity tag and the affinity matrix or weaken them to a lesser extent than the binding between the fusion protein and the interacting proteins and/or other substances. Elution by ionic strength equivalent to 0.35M KCl for 20 minutes is a good starting point for studying an uncharacterized ligand complex.

The method of the invention is suitable for detecting transient protein-protein interactions, and other transient interactions that involve proteins, including but not limited to DNA-protein interactions, RNA-protein interactions, carbohydrate-protein interactions, and lipid-protein interactions. The method of the invention is also suitable for detecting transient ligand-ligand interactions, where the ligand includes proteins, DNA, RNA, lipids and/or carbohydrates. For example, the method of the invention can be used to detect transient DNA-RNA interactions, DNA-DNA interactions, DNA-lipid interactions, DNA-carbohydrate interactions, RNA-RNA interactions, RNA-lipid interactions, RNA-carbohydrate interactions, lipid-lipid interactions, lipid-carbohydrate interactions and carbohydrate-carbohydrate interactions.

The invention contemplates the second ligand being a recombinant protein that contains at least one affinity tag. This fusion protein may be prepared using recombinant DNA methods. Accordingly, a nucleic acid molecule encoding the fusion may be incorporated in a known manner into an appropriate expression vector which ensures good expression of the fusion protein. Possible expression vectors include but are not limited to cosmids, plasmids, or modified viruses (e.g. replication defective retroviruses, adenoviruses and adeno-associated viruses), so long as the vector is compatible with the host cell used. The expression vectors are “suitable for transformation of a host cell”, which means that the expression vectors contain a nucleic acid molecule encoding the fusion protein and regulatory sequences selected on the basis of the host cells to be used for expression, which is operatively linked to the nucleic acid molecule. Operatively linked is intended to mean that the nucleic acid is linked to regulatory sequences in a manner which allows expression of the nucleic acid.

Suitable regulatory sequences may be derived from a variety of sources, including bacterial, fungal, viral, mammalian, or insect genes (For example, see the regulatory sequences described in Goeddel, Gene Expression Technology: Methods in Enzymology 185, Academic Press, San Diego, Calif. (1990)). Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector. It will also be appreciated that the necessary regulatory sequences may be supplied by the native protein of interest.

The recombinant expression vectors of the invention may also contain a selectable marker gene which facilitates the selection of host cells transformed or transfected. Examples of selectable marker genes are genes encoding a protein such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of the recombinant expression vectors. It will be appreciated that selectable markers can be introduced on a separate vector from the recombinant molecule.

The recombinant expression vectors encoding the fusion protein contain genes which encode at least one affinity tag fused in frame to the gene encoding the protein of interest. Typical fusion expression vectors include pGEX (Amrad Corp., Melbourne, Australia), pMal (New England Biolabs, Beverly, Mass.) and pRIT5 (Pharmacia, Piscataway, N.J.) which fuse glutathione S-transferase (GST), maltose E binding protein, or protein A, respectively, to the recombinant protein. The affinity tag may be fused directly or indirectly, such as through a peptide linker, to the recombinant protein.

Recombinant expression vectors can be introduced into host cells to produce a transformed host cell. The terms “transformed with”, “transfected with”, “transformation” and “transfection” are intended to encompass introduction of nucleic acid (e.g. a vector) into a cell by one of many possible techniques known in the art. Prokaryotic cells can be transformed with nucleic acid by, for example, electroporation or calcium-chloride mediated transformation. For example, nucleic acid can be introduced into mammalian cells via conventional techniques such as calcium phosphate or calcium chloride co-precipitation, DEAE-dextran mediated transfection, lipofectin, electroporation or microinjection. Suitable methods for transforming and transfecting host cells can be found in Sambrook et al. (Molecular Cloning: A Laboratory Manual, 2nd Edition, Cold Spring Harbor Laboratory press (1989)), and other laboratory textbooks.

Suitable host cells include a wide variety of eukaryotic host cells and prokaryotic cells. For example, the fusion protein may be expressed in yeast cells or mammalian cells. Other suitable host cells can be found in Goeddel, Gene Expression Technology: Methods in Enzymology 185, Academic Press, San Diego, Calif. (1991). In addition, the fusion protein may be expressed in prokaryotic cells, such as Escherichia coli (Zhang et al., Science 303(5656): 371-3 (2004)).

Yeast and fungi host cells suitable for carrying out the present invention include, but are not limited to Saccharomyces cerevisiae, the genera Pichia or Kluyveromyces and various species of the genus Aspergillus. Examples of vectors for expression in yeast S. cerevisiae include pYepSec1 (Baldari. et al., Embo J. 6:229-234 (1987)), pMFa (Kurjan and Herskowitz, Cell 30:933-943 (1982)), pJRY88 (Schultz et al., Gene 54:113-123 (1987)), and pYES2 (Invitrogen Corporation, San Diego, Calif.). Protocols for the transformation of yeast and fungi are well known to those of ordinary skill in the art (see Hinnen et al., Proc. Natl. Acad. Sci. USA 75:1929 (1978); Itoh et al., J. Bacteriology 153:163 (1983), and Cullen et al. (BiolTechnology 5:369 (1987)).

Mammalian cells suitable for carrying out the present invention include, among others: COS (e.g., ATCC No. CRL 1650 or 1651), BHK (e.g. ATCC No. CRL 6281), CHO (ATCC No. CCL 61), HeLa (e.g., ATCC No. CCL 2), 293 (ATCC No. 1573) and NS-1 cells. Suitable expression vectors for directing expression in mammalian cells generally include a promoter (e.g., derived from viral material such as polyoma, Adenovirus 2, cytomegalovirus and Simian Virus 40), as well as other transcriptional and translational control sequences. Examples of mammalian expression vectors include pCDM8 (Seed, B., Nature 329:840 (1987)) and pMT2PC (Kaufman et al., EMBO J. 6:187-195 (1987)).

Given the teachings provided herein, promoters, terminators, and methods for introducing expression vectors of an appropriate type into plant, avian, and insect cells may also be readily accomplished. For example, within one embodiment, the fusion protein can be expressed in plant cells (see Sinkar et al., J. Biosci (Bangalore) 11:47-58 (1987), which reviews the use of Agrobacterium rhizogenes vectors; see also Zambryski et al., Genetic Engineering, Principles and Methods, Hollaender and Setlow (eds.), Vol. VI, pp. 253-278, Plenum Press, New York (1984), which describes the use of expression vectors for plant cells, including, among others, PAPS2022, PAPS2023, and PAPS2034).

Insect cells suitable for carrying out the present invention include cells and cell lines from Bombyx, Trichoplusia or Spodotera species. Baculovirus vectors available for expression of proteins in cultured insect cells (SF 9 cells) include the pAc series (Smith et al., Mol. Cell Biol. 3:2156-2165 (1983)) and the pVL series (Lucklow, V. A., and Summers, M. D., Virology 170:31-39 (1989)). Some baculovirus-insect cell expression systems suitable for expression of the fusion protein are described in PCT/US/02442.

Alternatively, the fusion protein may also be expressed in non-human transgenic animals such as rats, rabbits, sheep and pigs (Hammer et al. Nature 315:680-683 (1985); Palmiter et al. Science 222:809-814 (1983); Brinster et al. Proc. Natl. Acad. Sci. USA 82:4438-4442 (1985); Palmiter and Brinster Cell 41:343-345 (1985) and U.S. Pat. No. 4,736,866).

In one embodiment, the fusion protein is expressed extrachromosomally and, optionally, the chromosomal copies of the gene (i.e. native gene encoding protein of interest) can be silenced. In another example, chromosomal copies of the gene of interest can be replaced by homologous recombination. Standard DNA recombinant techniques and yeast manipulation techniques are described in Sambrook, Fritsch, Maniatis, 1982 Molecular Cloning, Cold Spring Harbor Laboratory Press, and Current Protocols in Molecular Biology, John Wiley and Sons, Inc, New York, 1994.

Although the construction of affinity tags and expressing fusion proteins is not the essence of the present invention, several related issues deserve attention. When the organism is diploid, it is best to modify both chromosomal copies of the same gene. The affinity tag can be joined at the N-terminus or inside the protein or, preferably, at the C-terminus in order to avoid interfering with the expression from the natural promoter. Two different tags can be attached to two different subunits of the protein complex. Two different tags can be attached to either end of the same protein or to the same end of the protein. One tag can be attached to one end of the protein and another different tag can be inserted inside the gene. The binding domains of the affinity tag can be fused to the protein of interest directly or a by a linker sequence. The fusion protein can contain the whole protein of interest or only a part of it. It is best if the affinity tag is fused in such a way that it is accessible (i.e. can bind easily to the respective ligand) and does not affect the expression and folding of the protein and the assembly of a natural protein complex. In addition, the affinity tag should not interfere with the protein interactions and should be placed outside the interface. Since different organisms prefer different codons for the same amino acid, the sequences of the affinity tags can be modified to ensure good expression.

A person skilled in the art will appreciate that the recombinant nucleic acid expressing the fusion protein contains sequences ensuring its proper transcription, processing and translation. Preferably, the fusion protein is expressed under physiological conditions enabling its associations with other proteins or biomolecules in vivo.

In addition to the entire protein of interest, it will be appreciated that the fusion protein may contain only a portion of the protein of interest. The truncated or fragmented protein of interest may be prepared by expression of a truncated gene.

The scale of the experiment (number of the cells and the amount of total protein) depends on several factors: (a) the copy numbers of the ligand of interest and the interacting ligands, (b) the binding equilibrium constants of particular ligand interactions, (c) the sensitivity of the method that is used to identify the interacting ligands. By performing the method of the invention with different tagged subunits and with approximately 1011 cells, nearly all reported interacting proteins for RNA Polymerase II (approximately 30,000 copies per cell) were detected by mass spectrometry. The copy number of the protein of interest can be determined by Western blotting and the scale of the experiment can be recalculated.

When the protein of interest is present in low copy number in the cell, it can be expressed from a different promoter that generates more copies of the protein. Optionally, the promoter can be inducible and if the protein of interest is a subunit of a stable protein complex, the other subunits and the fusion protein of interest can be expressed from identical inducible promoters in order to ensure proper stoichiometries of the stable protein complex. In this case, great care should be taken not to over-express the protein of interest to a level that leads to formation of inclusion bodies.

The fusion protein can originate from one organism and the interacting proteins can originate from a different organism (e.g. host organism and parasite organism). The affinity tagged protein of interest can be exogenous or endogenous. However, the invention gives the most valid results when the fusion protein and the interacting proteins are expressed in their endogenous organism and their expression is driven from their natural promoters.

As stated above, the invention includes usage of affinity tagged proteins. It should be noted that the invention not only differs from other methods for purification via affinity tag but is even quite opposite to them (i.e. the greatest care must be taken to avoid the separation of the fusion protein from the affinity matrix during the elution). The novelty of the invention is apparent by comparison with the other methods for affinity purification as described by Bauer A. and Kuster B. in Eur. J. Biochem. Feb 2003, or, by comparison with the other methods for detection and analysis of protein interactions as described in a review by Phizicky E. and Fields S. in Microbiological Reviews, Mar. 1995, Vol. 95, No. 1.

Moreover, all basic methods for protein purification (i.e. affinity chromatography, hydrophobic interactions, ion exchange, reversed phase) include separation of the protein that has been bound to the solid phase. The invention offers a convenient way for separating substoichiometrically associated proteins and/or other biomolecules from the immobilized protein and constitutes a novel basic method for purifying proteins and detecting protein interactions.

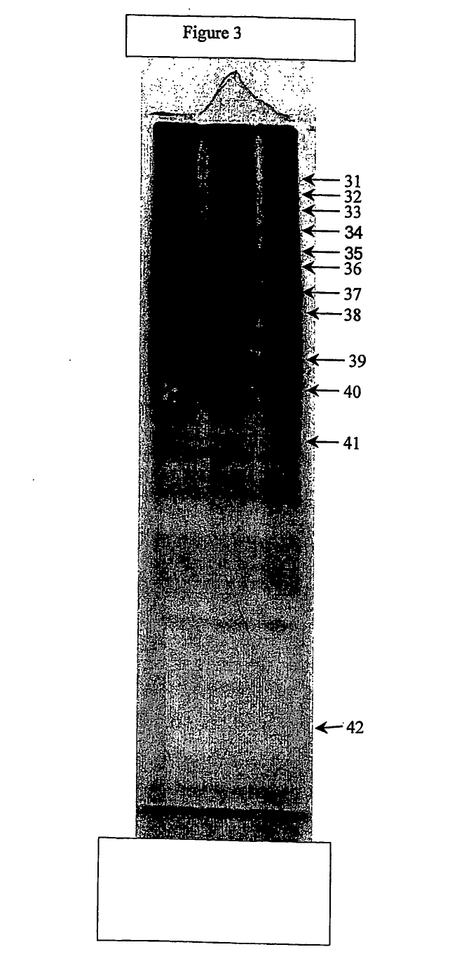

The invention can also be described as immobilizing selectively a complex containing the ligand of interest on a solid phase by binding between the ligand of interest and the solid phase and, after removing the unbound substances, separating the transiently associated ligand from the immobilized ligand of interest. The ligand of interest likely binds to the solid phase by dominantly one type of force (preferably, hydrophobic force) and elution of the transient ligand should be performed by weakening another force. As shown in the examples of Rpb1fused to a SpA-tag (see FIG. 3, 4 and 6), the ligand of interest (i.e. the fusion protein) binds to the affinity matrix (i.e. IgG beads) by hydrophobic force (i.e. Fc reactivity) and the transient ligand (i.e. transcription factors) are separated by weakening the electrostatic bonds (i.e. increasing the ionic strength of the medium).

As mentioned above, the method of the invention is not limited to separating, and optionally analyzing, protein-protein interactions. For example, a nucleoprotein complex can be immobilized by binding between its nucleic acid component and an affinity matrix coated with complementary sequence. The nucleic acid component of the complex can be genetically engineered so that it contains a Poly(G) and the complex can be immobilized on Poly(C) matrix (Sigma). The associated proteins can be eluted with increasing the ionic strength of the medium. In this case, the ligand of interest is bound to the affinity matrix by hydrogen bonds and the interacting proteins are separated by increasing the ionic strength of the medium.

In another example, separation of the transient protein interactor (first ligand) from the immobilized protein or protein complex (second ligand) can be achieved by enzymatic treatment (e.g. treatment that removes or adds an electrostatic charge as a result of acetylation or deacetylation or phosphorylation or dephosphorylation). In this case, essential information can be obtained about the nature of a particular protein-protein interaction.

The method of the invention is especially suitable for detecting proteins that interact with a multicomponent permanent protein complex. It is best if the elution is performed under such conditions that all subunits of the permanent protein complex remain substantially immobilized because a significant leakage into the liquid phase of any subunit, not only the tagged one, can suppress the identification and analysis of the substoichiometrically interacting proteins.

In a further example, the invention relates to methods of separating a first ligand from a second ligand in an affinity matrix system, wherein the first ligand associates in vivo with the second ligand, wherein the first ligand and the second ligand associate with each other by electrostatic forces, and wherein the second ligand is a recombinant fusion protein containing at least two different affinity tags, comprising the steps:
(a) introducing into a cell or organism a recombinant nucleic acid molecule encoding a fusion protein comprising the second ligand fused to at least two different affinity tags that can selectively bind to different affinity matrixes;
(b) expressing the fusion protein;
(c) obtaining a sample containing biological complexes that include the first ligand and the fusion protein;
(d) immobilizing the fusion protein on a first affinity matrix via a first affinity tag;
(e) removing unbound substances from the first affinity matrix;
(f) separating the fusion protein from the first affinity matrix;
(g) immobilizing the fusion protein on a second affinity matrix via a second affinity tag, which is different than the first affinity tag;
(h) removing unbound substances from the second affinity matrix;
(i) separating the first ligand from the immobilized fusion protein, which remains bound to the second affinity matrix during the separation, by decreasing the electrostatic forces between the first ligand and the fusion protein; and
(j) optionally, analyzing the separated first ligand.

In another example, the invention relates to methods of separating a first ligand from a second ligand in an affinity matrix system, wherein the first ligand associates in vivo with the second ligand, wherein the first ligand and the second ligand associate with each other by electrostatic forces, and wherein the second ligand is a protein complex containing two or more subunits of which are fused to different affinity tags, comprising the steps:
(a) introducing into a cell or organism recombinant nucleic acids molecules encoding fusion proteins comprising the two or more subunits of which are fused to different affinity tags that can selectively bind to different affinity matrixes;
(b) expressing the fusion proteins;
(c) obtaining a sample containing biological complexes that include the first ligand and the fusion proteins;
(d) immobilizing the fusion proteins on a first affinity matrix via a first affinity tag;
(e) removing unbound substances from the first affinity matrix;
(f) separating the fusion proteins from the first affinity matrix;
(g) immobilizing the fusion proteins on a second affinity matrix via second affinity tag, which is different than the first affinity tag;
(h) removing unbound substances from the second affinity matrix;
(i) separating the first ligand from the immobilized fusion proteins, which remain bound to the second affinity matrix during the separation, by decreasing the electrostatic forces between the first ligand and the fusion proteins; and
(j) optionally, analyzing the separated first ligand.

This two-step affinity purification using two different affinity tags fused to the ligand of interest or fused to two subunits of a ligand complex reduces the false positives by reducing the background from the contaminant proteins. In one example, the dual tags are the TAP (tandem affinity purification) tags containing a calmodulin binding domain and IgG binding domain separated by a Tev cleavage site. Other dual tags include, but are not limited to, a combination of GST and SpA, a combination of GST and SpG, a combination of GST and poly-His, a combination of MBP and SpA, a combination of MBP and SpG, a combination of MBP and poly-His, a combination of SBP and SpG, a combination of SBP and SpA, a combination of MBP and HA, and a combination of SBP and poly-His. A person skilled in the art will appreciate that different tags and different combinations of tags can be used.

Purification of interacting ligands by fusing two affinity tags to the protein of interest or to subunits of the protein complex of interest can be described as follows: (a) binding the recombinant protein(s) by the first tag to the first affinity matrix, (b) washing away the unbound proteins, (c) separating the fusion protein(s) from the first affinity matrix, (d) binding the fusion protein(s) to the second affinity matrix, (e) washing away the residual unbound proteins, (f) separating the interacting ligand from the fusion protein(s), which remains bound to the affinity matrix.

An affinity tag that remains bound to the respective affinity matrix at ionic strength equivalent to 0.4-0.5M KCl is suitable for carrying out the invention when the elution is performed by increasing the ionic strength. Affinity tags genetically derived from Streptococcal protein G (SpG) or from Protein A from Staphylococcus aureus (SpA) bind strongly to IgG-sepharose and are suitable affinity tags for carrying out the invention.

GST:glutathione binding is not affected by ionic strength equivalent to 0.5 M KCl and thus the GST-tag a good candidate for a single tag for carrying out the invention or a second tag when a dual affinity tag is used.

On the other hand, an affinity tag that can be separated from the respective ligand under conditions that do not disrupt the transient protein interactions can be used as a first tag. For example, GST-tag can be separated from glutathione beads by treatment with 5-20 mM reduced glutathione. Flag-tag can be separated by adding Flag-tag (DYKDDDDK) and can be used as a first tag.

MBP can be separated by treatment with 10 mM maltose and is suitable for a first tag. MBP:maltose binding is not affected by ionic strength that is equivalent to 0.7-1 M KCl and is suitable for a second tag or as a single tag.

Poly-His tag requires stringent conditions for release of the immobilized protein and is not suitable for a first tag. It can be used as a second tag or as a single tag because the tag remains bound to Ni2+ column at 0.5M KCl.

The Myc tag and hemagglutinin tag (HA) are suitable for a first tag. The HA-tag is more suitable for a second tag or single tag because it contains only two electrostatically charged amino acids.

Other tags that can be used for carrying out the invention are the T7-Tag sequence (the initial 11aa of the T7 gene), VSV-G (15 AA C-terminal peptide of VSV-G), b-GAL, Thioredoxin. The list illustrates but does not limit the scope of the invention. The review of K. Terpe in Appl. Microbiol. Biotechnol. 2003) describes popular affinity tags.

When the protein of interest is present in the cell in low copy number and an additional purification step is needed before the separation of the interacting ligands from the fusion protein, a combination of two different affinity tags can be used. It is best if the second immobilization is performed by the stronger tag, i.e. the dissociation constant (Kd) for the binding tag:ligand is smaller, in order to avoid leakage of the affinity tagged protein during the elution. Separation from the first solid support can be performed by proteolytic cleavage or by a treatment with a chemical agent.

The methods of the invention facilitate the identification and analysis of the first ligand that is associated with the second ligand in substoichiometric amounts. For example, the method of the invention solves the dynamic range problem of mass spectrometry, because the identification of the low abundance proteins (i.e. the first ligand) by mass spectrometry is facilitated if they are analyzed separately from the high abundance ones (i.e. second ligand).

In one example, naturally assembled RNA Polymerase II complexes can be immobilized on IgG-beads via a SpA-tagged subunit of the core complex. Then, the immobilized complexes can be treated with elution buffer containing 0.4 M KCl. This method separates the transcription factors into the liquid phase but the core complex remains immobilized on the IgG beads. The analysis of the transcription factors by mass spectrometry is greatly improved by the separation.

The inventors found that increasing the ionic strength does not disrupt either the bonds between the second ligand and the matrix (e.g. affinity tagged second ligand bound to the affinity matrix, such as a SpA:Ig bond) or the bonds between the subunits of permanent ligand complexes (e.g. the RNA Polymerase II core complex) but leads to rapid and almost complete dissociation of transient ligand complexes (e.g. transcription factors). Since the bonds between the second ligand and the affinity matrix are mainly hydrophobic in nature, the same must be the case for binding between the subunits of permanent ligand complexes (i.e. RNA Polymerase II). While, transient interactions between the transient ligand complexes are due in large part to electrostatic interactions, as demonstrated in the examples below.

Without being limited to theory, increasing the ionic strength destabilizes the electrostatic attraction between charged groups (salt bonds) because of the Debye-Huckel screening and according to Coulomb's law: F=q1q2/Dr2, where: F—strength of the electrostatic force, q—charge of the ion; D—effective dielectric constant of the media; r—distance between the ions. On the other hand, increasing the salt concentration stabilizes the hydrophobic bonds by competing out the water molecules from the non-polar patches of the protein surface so that they associate even stronger by hydrophobic interaction. This increase in the entropy of water molecules, as the number of them solvating hydrophobic surfaces decreases, is the main driving force for protein precipitation by “salting out” techniques (e.g. ammonium sulphate precipitation).

In addition, without being limited to theory, the fact that more than 60 interacting proteins can be dissociated from the RNA Polymerase II core complex with 12 subunits by weakening the electrostatic bonds (i.e. by increasing the ionic strength of the medium) allows the formulation of a general rule: “Permanent protein complexes are held together mainly by hydrophobic forces and transient ligand complexes are held together in large part by electrostatic attractions (mainly salt bonds between the charged amino acids)”. The salt bonds between the subunits of permanent ligand complexes are not affected by increasing the ionic strength because the ions cannot reach into the hydrophobic interface between the subunits and weaken them. The salt bonds that are formed during a transient ligand-ligand interaction are not shielded by the hydrophobic interface and they are easily disrupted by increasing the ionic strength of the medium. The other electrostatic attractions, which are much weaker, i.e. London forces, dipole-dipole, charge-dipole, are also weakened by increasing the ionic strength. The general rule above is a general rule and allows for some minor exceptions.

Jones, S and Thornton, J., PNAS 1996 showed by statistical analysis that the contact surfaces (interfaces) of transient protein complexes contain more hydrophilic residues than the contact surfaces of permanent protein complexes. The inventors also demonstrated that among all hydrophilic residues, the most important for formation of transient protein complexes are electrostatically charged ones (e.g. Lysine (positive), Arginine (positive), Aspartate (negative), Glutamate (negative) and partially Histidine (50% positively charged at pH 6.0)) that can form salt bonds or other electrostatic bonds. Another group of amino acids that are involved in transient protein-protein interactions are the ones that are electrostatically charged as a result of post-translational modifications (e.g. phosphorylation, acetylation).

Archakov et al. proposed, based on a statistical analysis (Archakov, A. et al. Proteomics, 2003), that formation of permanent complexes resembles and even might be a continuation of protein core folding, and henceforth, is due to the hydrophobic force. The inventors' results support this point of view and further suggest that stable protein complexes may have a common hydrophobic interior that is not accessible to the ions.

The nature of the transient protein-protein interactions was unknown prior to the inventors' findings. The inventors have found that transient protein-protein interactions are predominantly electrostatic. Moreover, since among all electrostatic forces charge-charge interactions (Coulomb force) are the strongest ones, they play a major role in transient protein interactions.

Without being limited by theory, three interesting features of the Coulomb force explain the inventors' finding that they are responsible for the formation of the majority of the transient protein complexes:

(I) Coulomb forces are long-range forces and they still play a role at 1 nanometer distance. All other forces are short-range forces, e.g. London forces occur when the molecules are close enough to induce electrostatic dipoles in one another.

(II) Coulomb forces are the strongest ones among noncovalent forces (up to −80 kcal/mol for salt bonds). The other noncovalent bonds are much weaker. Hydrogen bonds are −3 to −6 kcal/mol, Van der Waals bonds are −0.5 to −1 kcal/mol and hydrophobic bonds are −0.5 to −3 kcal/mol.

(III) Coulomb forces can be either attractive or repulsive, depending on the nature of the charges involved, i.e. attractive between positive and negative charges, and repulsive between two positive charges or between two negative charges.

Without being limited by theory, the inventors believe that the formation and disintegration of transient protein complexes is due to alternation of electrostatic attraction and electrostatic repulsion between the electrostatically charged amino acids. The switch could reflect the accomplishment of the function that requires formation of the transient protein complex and the three most plausible causes for the switch are: (a) change of orientation and/or position of the interacting proteins relative to one another, and/or (b) change of the conformation of one or both of the interacting proteins and/or (c) adding or removing an electrostatic charge as a result of post translational modification, e.g. acetyl group to Lysine or phosphate group to Serine, Tyrosine and Threonine.

Whatever the exact mechanism of transient protein-protein interactions, it seems clear that electrostatic forces are of primary importance. The invention is designed to detect transient protein-protein interactions by weakening the electrostatic forces and separating the interacting protein from the immobilized ligand of interest.

The invention has several significant advantages comparing to other methods for protein separation and/or detecting protein interactions.

(I) The method of the invention does not require a purified protein of interest for the preparation of affinity column,

(II) The immobilized protein or protein complex is in its natural modification state (e.g. phosphorylation state), which is very often necessary for proper formation of protein complexes,

(III) Since many proteins exist and interact as components of protein complexes, but not as single proteins, the invention is suitable for detecting transient protein interactions that involve multicomponent protein complexes.

(IV) The method of the invention detects proteins that have been associated with the fusion protein in vivo. Protein affinity chromatography according to Formosa T., Methods Enzymol. 1991, and GST pull downs are based on in vitro (de novo) association of the proteins with the immobilized protein and that makes possible many nonspecific interactions.

(V) The main advantage of the invention over protein purification via one or more affinity tags is the separation of the substoichiometrically interacting proteins from the tagged protein or protein complex and, as a result, greatly facilitated analysis and identification.

Different analytical methods can be used to identify and analyze the proteins that are purified by the method of the invention, e.g. mass spectrometry, enzymatic assays, immunodetection. Immunodetection can be used to analyze the interacting proteins and/or to verify the identity of the interacting proteins that cannot be identified unambiguously by mass spectrometry. When the proteins are eluted with ionic strength equivalent to 0.3M KCl or 0.4M KCl, they are not denatured and the eluate can be analyzed by biochemical assays for the presence of a particular enzymatic activity. Optionally, the eluate can be dialyzed against physiological buffer before the assay.

After building a proteomic map around the protein of interest (i.e. identifying unambiguously the interacting proteins), more sensitive methods for detecting the interacting protein can be used. For example, antibodies can be raised against particular interacting protein and it can be detected by immunodetection. However, one of the main utilities of the invention is the direct identification (preferably by mass spectrometry) of novel proteins that interact with a protein of interest in vivo.

After separating the interacting proteins in the liquid phase, they can be further fractionated by SDS-PAGE (the length of the gel depends on the complexity of the eluate) and localized by silver staining or Coomassie staining. Another variant is to run a native gel and separate different permanent complexes from one another.

The method of the invention is useful for detecting protein interactions in organisms where genetic approaches are impossible or very hard (e.g. Homo sapiens). The invention can also be used to elucidate the complete set of interactions that involve proteins having associations with human diseases and will reveal new targets for therapeutic intervention. Such diseases include, but are not limited to, Norrie disease; Alzheimer's disease; Parkinson's disease; beta3-adrenergic receptor gene mutation; achondroplasia; sickle cell anemia; thrombosis; alpha 1-antitrypsin deficiency; and the diseases listed in table 1.

Classic features of Norrie disease include specific ocular symptoms (pseudotumor of the retina, retinal hyperplasia, hypoplasia and necrosis of the inner layer of the retina, cataracts, phthisis bulbi), progressive sensorineural hearing loss, and mental disturbance, although less than one-half of patients are hearing impaired or mentally retarded. The Norrie gene encodes for a protein of 133 amino acids that has homologies at the C-terminus to a group of proteins including mucins. The high proportion of new mutations is an expected finding for an X-linked disorder with greatly reduced male reproductive fitness (Berger et al., 1992). It has been suggested that the norrin protein regulates vascularization of the cochlea and retina (Rehm et al, 2002).

Alzheimer's disease (AD) is characterized neuropathologically by progressive deposition of the 40-42 residue amyloid β-Protein (Aβ) in brain parenchyma and cerebral blood vessels. There are two basic types of Alzheimer's disease; the more common form, known as late-onset, strikes people, who are usually over the age of 65. This form of Alzheimer's is thought to be due to both genetic and environmental causes. The other form called autosomal dominant, early-onset or familial Alzheimer's, strikes people who are under the age of 65, and is strictly due to genetic abnormalities. This second form accounts for less than 10 percent of all Alzheimer's cases. Yet in these cases most of the major break through in discovering the chromosomal mutations that contribute to Alzheimer's disease are being discovered. Known mutations are listed in table 2.

There are similarities between Parkinson's disease and some other neurodegenerative disorders, especially Alzheimer's disease. For example, the production of Lewy bodies in Parkinson's disease is paralleled by the similar production of senile plaques in Alzheimer's disease (and to some extent in normal aging). The protein that is altered in familial Parkinson's disease has similarities to the non-beta amyloid component of Alzheimer's disease. Known mutations for Parkinson's disease include C418R; E46K; N279K and R98Q.

The Beta3-adrenergic receptor gene makes a protein in fat cells that is involved in determining how much fuel your body burns when you are resting. A mutation in this gene slows down how quickly a person burns fat and thus increases their tendency to be obese. One specific mutation in this gene, called TRP64ARG, is almost four times more common in Pima Indians than in people of European descent, and is one and a half times more common in people of African or Mexican descent. The prevalence of the TRP64ARG gene mutation in these populations probably accounts at least in part for why these ethnic groups have a higher rate of Type 2 diabetes.

Achondroplasia, the most common genetic form of dwarfism, is an autosomal dominant disorder whose underlying mechanism is a defect in the maturation of the cartilage growth plate of long bones. Achondroplasia has recently been shown to result from a Gly to Arg substitution in the transmembrane domain of the fibroblast growth factor receptor 3 (FGFR3), although the molecular consequences of this mutation have not been investigated. By substituting the transmembrane domain of the Neu receptor tyrosine kinase with the transmembrane domains of wild-type and mutant FGFR3, the Arg380 mutation in FGFR3 is shown to activate both the kinase and transforming activities of this chimeric receptor. Residues with side chains capable of participating in hydrogen bond formation, including Glu, Asp, and to a lesser extent, Gln, His and Lys, were able to substitute for the activating Arg380 mutation. The Arg380 point mutation also causes ligand-independent stimulation of the tyrosine kinase activity of FGFR3 itself, and greatly increased constitutive levels of phosphotyrosine on the receptor. These results suggest that the molecular basis of achondroplasia is unregulated signal transduction through FGFR3, which may result in inappropriate cartilage growth plate differentiation and thus abnormal long bone development. Achondroplasia may be one of the number of cogenital disorders where constitutive activation of a member of the FGFR family leads to development abnormalities.

Sickle cell anemia is one of the first illness to be understood at the molecular level. In this genetic disease, Glu is replaced by a Val in the beta globin chain. A single base substitution brings about this change. This is a nasty substitution because it replaces an acidic amino acid with a hydrophobic sidechain. The Val fits nicely into a hydrophobic pocket in another beta globin subunit causing a polymerization between beta chains. This creates a kind of domino effect leading to the formation of long fibers made of many millions of damaged hemoglobulin molecules. The Hb polymers cause the cells to distort into a sickle shape. The mutation is lethal for homozygotes but the mutation persists in populations with a high incidence of malaria. This is generally thought to be because heterozygotes have a higher resistance to malaria. The malaria parasite spends some of its life in the red blood cell and these sickle cells are not a good host.

Thrombosis is a serious condition, killing hundreds of thousands and debilitating millions each year by myocardial infraction, pulmonary embolism, or stroke. Inherited thrombophilia is a genetically determined tendency to venous thromboembolism. The term reflects the presence of an inherited factor that, per se, predisposes towards thrombosis, but which, because of the episodic nature of thrombosis, requires interaction with other factors (inherited or acquired) before onset of the clinical disorder. Mutations in genes encoding proteins that activate coagulation pathways or inactivate anticoagulation mechanisms play an important role in the predisposition to venous thrombosis. Blood coagulation is downregulated by the protein C anticoagulant system. Protein C is activated on endothelial cells by thrombin bound to thrombomodulin. Together with its cofactor, protein S, activated protein C degrades the activated forms of factor V and VIII. Resistance to activated protein C is considered the most prevalent inherited cause of venous thrombosis. The most common cause of this resistance is a point mutation in the factor V gene (Factor V Leiden) that replaces an arginine residue at position 506 with a glutamine, R506Q (G to A transition at position 1691). This change causes the mutant factor Va to resist proteolysis by activated protein C. The frequency of the Leiden genetic variation is relatively high in Caucasian populations (up to 6%) but it is much lower in African and Asian populations (down to 0%). Prothrombin (factor II) is the precursor of thrombin, the final effector of the clotting cascade that leads to the formation of fibrin. Prothrombin potentiates coagulation by positive feedback loops and also promotes anticoagulation by the protein C pathway. A G to A mutation at nucleotide 20210 which is the last residue at the 3'-untranslated region of the prothrombin gene has been found to be associated with higher plasma concentrations of prothrombin and a 2.8-fold increased risk of venous thrombosis. The prothrombin 20210 G to A mutation carrier frequency is highest among whites of Southern European origin (3%), slightly lower among whites of Northern European origin (1.7%), uncommon among persons of Africa (0.7%) and very uncommon among persons of Asian origin (0%).

Hyperhomocystinaemia has also been shown to be a risk factor for venous thrombosis among other conditions. The most common genetic cause of hyperhomocystinaemia is an amino acid substitution in methylenetetrahydrofolate reductase (MTHFR), the enzyme that converts 5,10-methylenetetrahydrofolate to 5-methyltetrahydrofolate, the methyl donor in the conversion of homocysteine to methionine. A point mutation which changes a C to T at nucleotide 677 results in the substitution of valine to alanine at position 222 (C677T). The mutant enzyme exhibits reduced activity. Homozygous mutation in MTHFR is associated with a 3-fold increase in risk for premature cardiovascular disease. The frequency of this genetic variant is relatively high in French Canadian, European, Middle Eastern and Japanese populations (12-15%), lower in Finnish (5.4%) and Dutch (5.2%) populations, and very low in African-American populations (1.4%).

By far the most prevalent type of clinically important alpha 1-antitrypsin (AAT) deficiency is classified as phenotype Pi Z. AAT deficiency is the most prevalent potentially lethal hereditary disease of Caucasians and was discovered by Laurell and Eriksson in 1963. One of the amino acid substitutions (Glu342→Lys342) may result in misfolding of the AAT, leading to intracellular accumulation and intracellular degradation of the abnormal protein (Sifers, R. N., Finegold, M. J., and Woo, S. L. C. 1989. Alpha-1-antitrypsin deficiency: Accumulation or degradation of mutant variants within the hepatic endoplasmic reticulum. Am. J. Respir. Cell Mol. Biol. 1:341-345). The structural alteration in the Z variant appears to allow “loop-sheet polymerization” of the molecule, during which the reactive center loops of one molecule become inserted into an opening in the A sheet of another molecule (Lomas, D. A., Evans, D. L., Finch, J. T., and Carrell, R. W. 1992. The mechanism of Z alpha-antitrypsin accumulation in the liver. Nature 357:605-607, Lomas, D. A., Evans, D. L., Stone, S. R., Chang, W.-S. W., and Carrell, R. W. 1993. Effect of the Z mutation on the physical and inhibitory properties of α1-antitrypsin. Biochemistry 32:500-508). The S variant: The S variant has a single amino acid substitution (Glu264→Val264) when compared with the most prevalent normal type of AAT. The S mutation is not associated with intracellular accumulation of the protein, and the S protein inhibits elastase normally. The amounts of the S protein that reach the circulation are slightly lower than normal, because of intracellular degradation of the AAT before it is secreted (Curiel, D. T., Chytil, A., Courtney, M., and Crystal, R. G. 1989. Serum α1-antitrypsin deficiency associated with the common S-type (Glu-Val) mutation results from intracellular degradation of al-antitrypsin prior to secretion. J. Biol. Chem. 264:10477-10486). The S allele is slightly more prevalent than the Z allele among U.S. Caucasians, and it is much more prevalent in the Iberian peninsula and neighboring countries. Individuals with the Pi S phenotype do not appear to be at increased risk for lung or liver disease.

Overo lethal white syndrome (OLWS) is an inherited syndrome of foals born to American Paint Horse parents of the overo coat-pattern lineage. Affected foals are totally or almost totally white and die within days from complications due to intestinal aganglionosis. Related conditions occur in humans and rodents in which mutations in the endothelin receptor B (EDNRB) gene are responsible. EDNRB is known to be involved in the developmental regulation of neural crest cells that become enteric ganglia and melanocytes. There is a polymorphism in the equine EDNRB gene closely associated with OLWS. This Ile to Lys substitution at codon 118 is located within the first transmembrane domain of this seven-transmembrane domain G-protein-coupled receptor protein.

The method of separating and optionally analyzing ligands that form transient complexes with other ligands can be used in drug or pre-drug screening assays. For example, the eluate obtained from the method of the invention described above, can be analyzed for the presence of substances that can be drugs or pre-drugs. In addition, a drug library can be applied to the living cells and/or the cellular lysate, and then the eluate can be analyzed for the presence of one or more members of the library. Also, after the elution of the interacting proteins, the immobilized protein or protein complex can be used for screening libraries of chemicals for a particular chemical that binds to it. Methods for drug discovery are described by Rossi D. and Sinz M., Marcel Dekker 2001 “Mass Spectrometry in Drug Discovery”.

Moreover, the invention can be used as a novel method for drug design and drug discovery. A chemical can be identified as a drug or a pre-drug by its capability to affect the formation and/or disintegration of a particular protein complex that is associated with a disease. Such a chemical should have two characteristic features: (a) it should affect selectively a particular protein interaction (or group of interactions involving a protein that is associated with a disease), unlike increasing the ionic strength which affects transient protein interactions nonselectively, and (b) it should affect the particular protein interaction at a concentration below 10 mM, i.e. a concentration that does not change significantly the ionic strength. After the chemical or a biomolecule is identified as a pre-drug, it can be further modified and the experiment can be repeated with lower concentrations of the said chemical or biomolecule. This can be used to obtain such a chemical that affects selectively the formation and/or disintegration of the protein complex at micromolar concentration, i.e. concentration that is appropriate for treatment of human subjects.

The effect of the drug or pre-drug can be determined by monitoring the presence and/or the concentration of the interacting proteins in the eluate. Preferably, after building a proteomic map around the protein of interest (i.e. identifying unambiguously the interacting proteins), other methods, that are more sensitive than mass spectrometry, can be used. For example, antibodies can be raised against a particular protein, and then it can be detected by immunoassays. Also, the interacting proteins can be tagged and detected by antibodies against the tag.

The effect of the pre-drug on a particular protein interaction can be tested in several ways:
(i) The pre-drug can be added to the growing cells, the lysis buffer and the washing buffer. In this case, the elution is performed with washing buffer without a pre-drug. Alternatively, the elution is performed with washing buffer with increased ionic strength but without a pre-drug.
(ii) When the chemical is applied to the immobilized protein complex after the removal of unbound substances, it can be used as a single elution agent or together with one or more nonspecific elution agents, i.e. the elution can be done with the chemical in the washing buffer or with the chemical in the elution buffer.
(iii) The high salt eluate, optionally dialyzed against washing buffer or other physiological buffer, can be recombined with the immobilized fusion protein and the pre-drug and incubated for 10-60 minutes or more, depending on the concentration of the proteins and the equilibrium binding constant of the protein interaction. The restoration of the protein complex can be determined by monitoring the amount of one or more interacting proteins in the eluate and/or on the affinity matrix (i.e. after the incubation and the removal of unbound proteins, the elution is performed with high salt buffer).

A method for drug design and drug discovery that is based on the finding that the nature of transient protein-protein interactions is dominantly electrostatic contains the following steps:
(i) Selecting a protein or a protein complex associated with a disease and obtaining a cell line or organism with an affinity tagged gene(s). According to our finding, a disease that is caused by a mutation that changes the electrostatic properties of a protein is due to an aberration in a transient protein interaction. The mutation can change the electrostatic properties of the protein by replacing Lysine and/or Arginine and/or Aspartate and/or Glutamate and/or Histidine with an uncharged or oppositely charged amino acid. In addition, a mutation can change the electrostatic properties of the protein by replacing other amino acids with Lysine and/or Arginine and/or Aspartate and/or Glutamate and/or Histidine. See Table 1 and Appendix 1, and note how many severe diseases are caused by such mutations.
(ii) Performing the method of the invention and/or other affinity purifications in order to sort out which group of proteins are associated in stable protein complexes (i.e. associated mainly by hydrophobic forces) and which ones are associated in transient complexes (i.e. associated mainly by electrostatic forces). Building a proteomic map (i.e. map of protein-protein interactions) around the protein that is associated with the disease.
(iii) Performing the method of the invention under different conditions (functional proteomics) and/or with cell lines originating from organism(s) with a disorder. Determining which protein interaction is associated with the specific disease and determining the structure and the composition of the interface (the part of a protein surface that is interacting with the other protein).
(iv) Designing and/or synthesizing a chemical or a group of chemicals capable of binding to the mutant protein and restoring, at least partially, its electrostatic properties. Alternatively, the drug may be designed to bind to the native protein interactor and modify its electrostatic properties in such a way that it can interact with the mutant protein. An already existing drug or chemical can be used in the beginning and later can be modified. The drug should consist of two parts—(I) Part that binds selectively to the protein and ensures the specificity of the drug. Methods for designing and/or synthesizing chemicals that bind selectively to particular protein structure (most often by shape complementarity) are described in the literature and known to persons skilled in the art—Pong S., Biopharmaceutical Drug Design and Development, Blackwell Publishing 1999, (II) Part that is electrostatically charged and restores, at least partially, the electrostatic properties of the protein. For example, if a mutation is replacing a positively charged amino acid (Lysine or Arginine) with uncharged one, the drug should contain a positively charged group and after the binding of the drug to the protein, such group should be located at the same place as the mutated amino acid or close to it. Designing a drug in such a way that it binds selectively to a mutant protein and brings in an electrostatic charge that modifies the electrostatic properties of the mutant protein is a novel approach for drug design.
(v) Testing the capability of the chemical (pre-drug) to affect the proper formation and/or disintegration of the protein complex that is associated with a disease.

The following non-limiting examples are illustrative of the present invention:

### EXAMPLES

### Example 1

### Rpb1-SpA-tag Fusion

Heterologous DNA encoding a selectable marker and a SpA-tag fused in-frame to the carboxy-terminus of Rpb1 was constructed and administered into Saccharomyces cerevisiae cells. The resulting strain expresses a fusion protein containing Rpb1 and a SpA-tag containing four IgG-binding domains. After selecting the transformants, the proper expression of the fusion protein was checked by Western blofting. Cell cultures were grown in YPD medium at 30° C. and constant vigorous shaking and cells were collected at OD600=1.2. The following protocol was used:
1. Cells were collected by centrifugation at 4,000 rpm for 4 minutes and the medium was decanted. All subsequent manipulations were performed on ice or at 0° C. to 5° C.
2. Cells were suspended in 200 ml water and distributed into 50 ml plastic tubes; centrifuged at 4,000 rpm for 4 minutes and the water was decanted.
3. Frozen in liquid nitrogen for 1 minute.
4. The solid pellet was broken, and protease inhibitors and 2-3 pieces (around 8 cm³) of dry ice were added; ground with a coffee grinder for 1 minute; the powder was transferred to a beaker and, after allowing it to melt, the same volume of 2×Lysis buffer was added. and the lysate was stirred with a plastic rod for 2 minutes. 2×Lysis Buffer: 200 mM KCl, 50 mM HEPES pH 7.4, 20% glycerol, 2% DMSO, 0.2% TritonX100, 2 mM PMSF, 2 mM benzamidine, 2×phosphatase inhibitors.
5. Centrifuged at 16,000 rpm for 30 minutes and the clarified lysate was transferred to a clean tube.
6. 800 microliters of IgG beads was added and the tube was rotated for 2 hours.
7. Centrifuged at 3,000 rpm for 2 minutes and the supernatant was decanted.
8. The IgG beads were transferred and distributed to four parallel 10 ml chromatography columns. Each column was washed with 10 ml washing buffer (WB) by applying 200 microliter aliquots. WB: 100 mM KCl, 10 mM HEPES pH 7.4, 5% glycerol, 1% DMSO, 0.1% TritonX100.
9. The beads were transferred to a 5 ml tube and 3 ml of WB containing 0.4M KCl (WB (0.4M)) was added.
10. The tube was rotated for 10 minutes.
11. The beads and the buffer were transferred to a 10 ml chromatography column and the eluate was collected by gravity flow. The beads were washed with two column volumes of WB (0.4M) and the eluates were combined.
12. The eluate was distributed to eppendorf tubes and ⅕ volume of 100% TCA was added.
13. Incubated on ice for 1 hour; centrifuged for 1 hour at 14,000 rpm at 0° C. to 5° C.
14. The supernatant was decanted and 1 ml 90% acetone was added. Vortexed for 30 seconds and centrifuged for 10 minutes at 14,000 rpm. The supernatant was decanted.
15. Dried with a SpeedVac for 15 seconds.
16. Protein gel electrophoresis (SDS-PAGE—10% gel, 20 cm long) and silver staining were performed.

A gel image of high salt eluate from immobilized RNA Polymerase II from Saccharomyces cerevisiae is shown in FIG. 3. The digits and arrows at the right side of the gel indicate the identified proteins. Legend: 31—Spt6, 32—Rgr1, 33—Spt6+Spt5, 34—Tfg1+Spt5, 35—Set3, 36—Cet1+Med1, 37—Pob3, 38—Npl3, 39—Tfg2+Iws1, 40—Cdc73, 41—TFIIS, 42—Ess1.

Protein identifications by mass fingerprinting were made essentially as described in Shevchenko A et.al. Anal. Chem., 1996, and Van Montfort, B. A. et.al., J. Mass Spectrom., 2002. After the SDS-PAGE and silver staining the gel lanes were cut across the whole length (including the bands stained most weakly with silver and “blank” regions) and the proteins in the gel pieces were reduced by DTT treatment and alkylated by iodoacetamide treatment. After trypsin digestion, the resulting peptides were purified and concentrated by reverse phase chromatography on C18-matrix. The peptides were co-crystallized with CHCA (a-cyano-4-hydroxycinnamic acid) ionization matrix and subjected to MALDI-TOF MS. After obtaining the MALDI-TOF MS spectra, the proteins were identified by matching the list of experimentally obtained peptide masses with computer-generated lists of peptide masses for every predicted protein in a Saccharomyces cerevisiae databanks. When contaminant proteins were identified in the sample, the corresponding peptide masses were subtracted and the search was performed again with the remaining masses. When a band is not indicated by an arrow, it means that it contains alternative translation products of proteins that are indicated elsewhere or their degradation products or contaminant proteins. The proteins identified with high confidence are listed in the text (the legends) and indicated in the Figures.

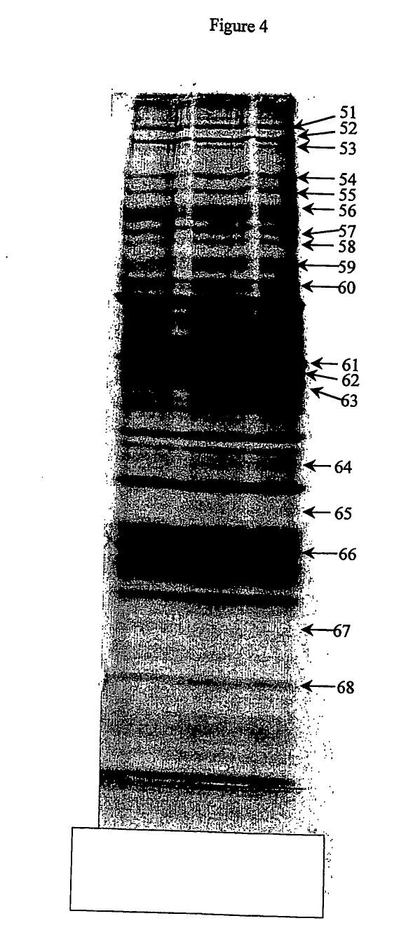

A gel image of high salt eluate from immobilized RNA Polymerase II from Soccharomyces cerevisiae is shown in FIG. 4. The fusion protein contains Rpb1 and a SpA tag containing two IgG binding domains. 18 liters culture was grown to OD600=0.7 and the separation of the interacting proteins was performed with washing buffer containing 0.4 M KCl (WB(0.4M)). The following protocol was used:
1. The cells were collected by centrifugation at 4,000 rpm for 4 minutes and the medium was decanted. All subsequent manipulations were performed on ice or at 0° C. to 5° C.
2. The cells were suspended in 200 ml water and distributed into 50 ml plastic tubes; centrifuged at 4,000 rpm for 4 minutes and the water was decanted.
3. Frozen in liquid nitrogen for 1 minute.
4. The solid pellet was broken, and protease inhibitors and 2-3 pieces (around 8 cm³) of dry ice were added; ground with a coffee grinder for 1 minute; the powder was transferred to a beaker and, allowed to melt, the same volume 2×Lysis buffer was added. Stirred with a plastic rod for 2 minutes. 2×Lysis Buffer: 200 mM KCl, 50 mM HEPES pH 7.4, 20% glycerol, 2% DMSO, 0.2% TritonX100, 2 mM PMSF, 2 mM benzamidine, 2×phosphatase inhibitors.
5. Centrifuged at 16,000 rpm for 30 minutes and transferring the clarified lysate to a clean tube.
6. 800 microliters of IgG beads were added and the tube was rotated for 2 hours.
7. Centrifuged at 3,000 rpm for 2 minutes and the supernatant was decanted.
8. The IgG beads were transferred and distributed to four parallel 10 ml chromatography columns. Each column was washed with 10 ml washing buffer (WB) by applying 200 microliter aliquots. WB: 100 mM KCl, 10 mM HEPES pH 7.4, 5% glycerol, 1% DMSO, 0.1% TritonX100.
9. The beads were transferred to a 5 ml tube and 30 ml WB was added containing 0.4M KCl (WB (0.4M)).
10. The tube was rotated for 10 minutes.
11. The beads and the buffer were transferred to a 10 ml chromatography column and the eluate was collected by gravity flow. The beads were washed with two column volumes of WB (0.4M) and the eluates were combined.
12. The eluate was distributed to eppendorf tubes and ⅕ volume of 100% TCA was added.
13. Incubated on ice for 1 hour; centrifugation for 1 hour—14,000 rpm at 0° C. to 5° C.
14. The supernatant was decanted and 1 ml 90% acetone was added. Vortexed for 30 seconds and centrifuged for 10 minutes at 14,000 rpm. The supernatant was decanted.
15. Dried with a SpeedVac for 15 seconds.
16. Protein gel electrophoresis (SDS-PAGE—10% gel, 20 cm long) and silver staining were performed.
   Legend: 51—Spt5+Spt6, 52—Spt5+Spt6, 53—Spt5+Spt6, 54—Ctr9+Spt5+Spt6, 55—Spt5+Ydl145 (Cop1)+Spt6, 56—Tfg1+Spt5+Spt6+Spt16, 57—Spt5+Tfg1+Sec21, 58—Spt5+Tfg1, 59—Set2+Spt5+Rff1+Ydl145 (Cop1), 60—Cet1, 61—Tfg2+Iws1, 62—Ceg1, 63—Cdc73, 64—TFIIS, 65—Rtt103, 66—Tfg3, 67—Yhr121w, 68—Ess1.

### Example 2

### Nonspecific Binding to IgG

Besides the separation of the interacting proteins and other biomolecules from the immobilized fusion protein, increasing the ionic strength causes separation of residual contaminant proteins from the affinity matrix. One way to avoid false signals (i.e. eliminate the background from the contaminants) is to perform the method of the invention with cellular lysate from an organism devoid of affinity tagged protein and identify the proteins in the eluate as contaminants. Alternatively, the proteins that are isolated by performing the method of the invention with several biologically unrelated proteins can be compared and the common ones can be identified as contaminants. The contaminants are not indicated in the Examples.

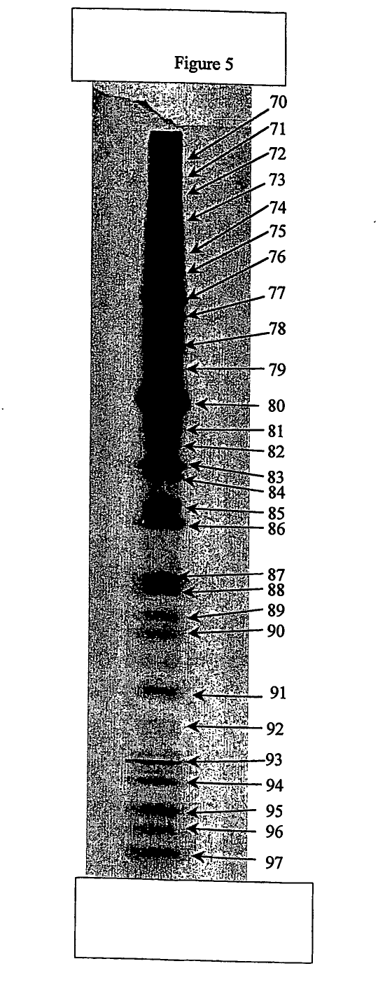

An example of identifying proteins that bind nonspecifically to IgG beads and/or to other proteins (contaminants) is shown in FIG. 5. Protein extract from Saccharomyces cerevisiae lacking an affinity tagged protein was incubated with IgG beads, the unbound proteins were removed and proteins that bind to IgG-sepharose and/or to other proteins nonspecifically were eluted with high salt in the washing buffer. The same protocol as in FIG. 4 was used. Legend: 70—Acc1+Fas1+Fas2, 71—Lys2+Ydr098, 72—Mdn1+Tra1, 73—Sec27+Ade3, 74—Prt1+Lys2, 75—Sse1+Lsg1, 76—Ssb1+Ssa2+Pab1+Ded1, 77—Hsp60+Dhh1, 78—Gcd11+Ded1+Nop12+Nog2, 79—Tef2+Phr1, 80—Tef2+L3, 81—Nop3+Tef2, 82—Yor060+Ydj1, 83—Stm1+Rpl4, 84—Tif34, 85—Rpp0+Rpl5, 86—Tef2+Nop1, 87—Rps1+Rpl2, 88—Rps3+Rpl8, 89—Rps2, 90—Rps5, 91—Rps8+Rpl9, 92—Rpl10, 93—Rps20, 94—Rpl20, 95—Rps14, 96—Rps17, 97—Rpl27+Rps24. The following contaminants are not indicated in the other figures: Phr1, Tef2, Nop1, Acc1, Nop3, Fks1, Yor060, Fas2, Ydj1, Fun12, Stm1, Aro1, Rpl4, Lys2, Tif34, Ydr098, Rpp0, Rpg1, Rpl5, Rps3, Ade3, Rps1b, Sec27, Rpl2, Prt1, Rpl8, Tef2, Rps2, Lsg1, Rps5, Dhh1, Rpl10, Ded1, Rpl9, Ssz1, Hsp60, Rps8, Gcd1, Rps20, Nop12, Rpl20, Nog2, Pab1, Rps17, Fas1, Ssb1, Ssa2, Sse1, Rpl27, Rps14, Rps24, Hfa1, Zuo1, Asc1, Mdn1, Tra1, Dyn1, Myo2, Ira2.

### Example 3

### Higher Salt Eluates

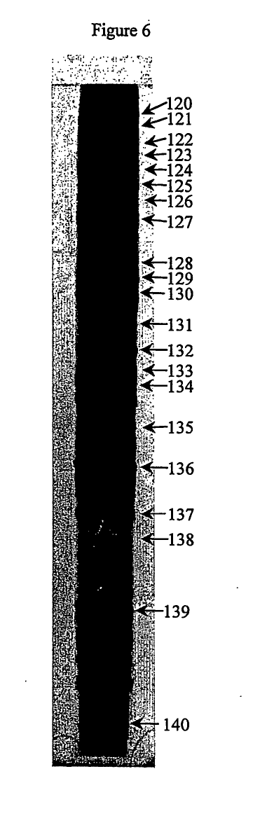

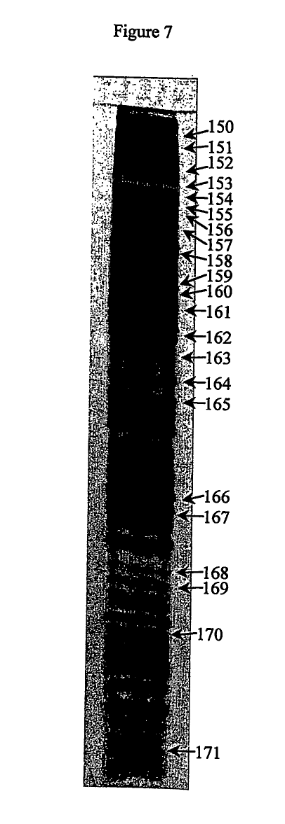

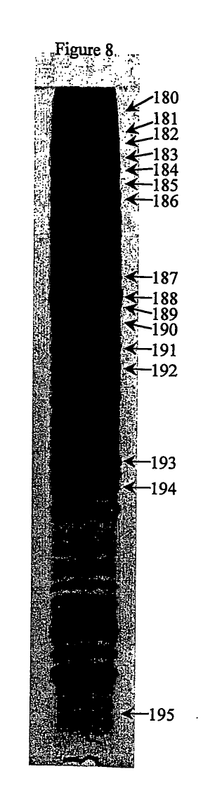

After identifying the contaminants for a particular organism, the possibility to assign a contaminant protein as a true interactor, i.e. to produce a false positive interactor, are minimal and the experiments can be performed under less stringent conditions. For example, after immobilizing the complexes on the affinity matrix, the removal of the unbound proteins can be very brief (i.e. washing 200 mcl IgG beads with 6 ml washing buffer) and the elution of the interacting proteins can be performed by a higher salt concentration (i.e. 0.7 M potassium acetate). High salt eluates from immobilized RNA Polymerase II complexes from Saccharomyces cerevisiae obtained in three different experiments from three different strains each expressing different SpA-tagged subunit are shown in the following Figures. SpA-tags contain two IgG binding domains. The fusion protein in FIG. 6 contains Rpb1, the fusion protein in FIG. 7 contains Rpb3, the fusion protein in FIG. 8 contains Rpb9. The eluates were resolved by SDS-PAGE and the proteins were identified by MALDI-TOF MS.

Legend for FIG. 6: 120—Spt6, 121—Spt6, 122—Spt5+Cyr1+Clu1, 123—Tfg1+Nam7+Spt16, 124—Fcp1+Cdc48+Sec21, 125—Taf5+Top2, 126—Cet1+Pcf1+Prp6, 127—Pob3, 128—Rtt103+Ptc3+Has1, 129-Tfg2+Iws1+Pap1, 130-Tfg2+Iwr1; 131-Rai1, 132-Sua7, 133—TFIIS, 134—Ypl253, 135—Ssn3+Mrt4, 136—Tfg3+Ptc1, 137—Med7, 138—Yhr121w, 139—Mbf1+Taf9+Yor379c, 140—Ybr262c+Ybr174c.

Legend for FIG. 7: 150—Spt6, 151—Spt5+Ctr9+New1, 152—Spt16+Tor2, 153—Tfg1+Hpr5+Nam7, 154—Fcp1+Cdc48+Nam7, 155—Fcp1+Rad3, 156—Fcp1+Taf5+Pbp1, 157—Cet1+Cyr1, 158—Pob3, 159—Rtt103+Ptc3, 160—TFIIH+Rad23+Drs1, 161—Tfg2+Iws1, 162—Rai1+Pob3+Rgr1+Ygr086c, 163—Fun11+Sub1, 164—TFIIB, 165—TFIIS+Yol045, 166—Tfg3, 167—Din7, 168—Yhr121w, 169—Nhp2, 170—Mbf1.

Legend for FIG. 8: 180—Spt6+Stt4+Ydl145 (Cop1), 181—Spt5+New1+Tof1, 182—Spt5+Rad3, 183—Tfg1+Cdc48+Rgr1+Nam7, 184—Fcp1+Cdc48+Rad1+Sec21, 185—Fcp1+Sup35+Rap1, 186—Cet1+Rap1+Cdc6, 187—Tfg2+Iws1, 188—Ceg1+Kin1+Prp8, 189—Ceg1+Rgm1, 190—Ygr086c+Ydr065w, 191—TFIIB, 192—TFIIS+Tho1+Bim1, 193—Tfg3+Tfa2 (TFIIE), 194—Set3+Tfb2 (TFIIH)+Yol022c. Cdc6, Prp2, Prp16, Rif1 and Cft1 were also detected.

### Example 4

### Additional Purification Step to Reduce False Positives

Yet another way to reduce the possibility of producing false positives is to reduce the background from the contaminant proteins by performing an additional purification step before the separation of the interacting proteins from the fusion protein. Preferably, the additional step is an affinity purification step and in this case, two different affinity tags can be fused to the protein of interest (i.e. protein is fused to a dual tag) or to two subunits of a protein complex, and, after the second immobilization, the interacting proteins are separated from the immobilized fusion protein. The tag used in the following experiments is a modified version of the TAP-tag created by Ghaemmaghami, S., et.al. Nature, 2003.

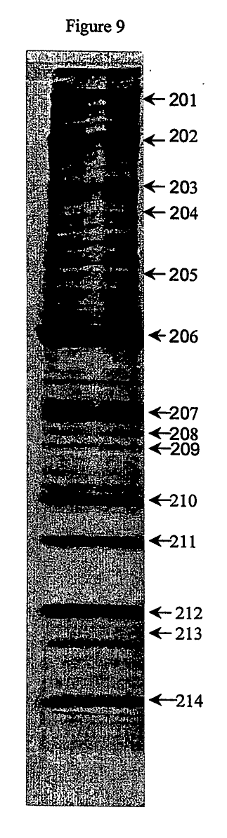

A gel image of high salt eluate from immobilized TFIIF from Saccharomyces cerevisiae is shown in FIG. 9. The carboxy-terminus of the chromosomal copy of the gene encoding Tfg1 is fused in-frame with a TAP-tag. 18 liters Saccharomyces cerevisiae culture was grown in YPD medium to OD600=0.7. The complex was purified on IgG beads and further immobilized on calmodulin beads. The elution was performed with 0.3 M KCl in the buffer.

Legend: 201—Rpb1, 202—Rpb1+Rpb2, 203—Fcp1+Rpb2+Rpb1, 204—Rpb1+Rpb2+Fcp1, 205—Glo3, 206—Rpb3, 207—TFIIS, 208—Mpd2, 209—Rpb4, 210—Yhr121w+Rpb5, 211—Rpb6, 212—Rpb7, 213—Mbf1+Rpb8, 214—Rpb9.

The following protocol was used:
1. The cells were collected by centrifugation at 4,000 rpm for 4 minutes and the medium was decanted. All subsequent manipulations were performed on ice or at 0° C. to 5° C.
2. The cells were suspended in 200 ml water and distributed into 50 ml plastic tubes; centrifuged at 4,000 rpm for 4 minutes and the water was decanted.
3. Frozen in liquid nitrogen for 1 minute.
4. The solid pellet was broken and protease inhibitors and 2-3 pieces (around 8 cm³) of dry ice were added; ground with a coffee grinder for 1 minute; the powder was transferred to a beaker and, after allowing it to melt, the same volume of 2×Lysis buffer was added. Stirred with a plastic rod for 2 minutes. 2×Lysis Buffer: 200 mM KCl, 50 mM HEPES pH 7.4, 20% glycerol, 2% DMSO, 0.2% TritonX100, 2 mM PMSF, 2 mM benzamidine, 2×phosphatase inhibitors.
5. Centrifuged at 16,000 rpm for 30 minutes and the supernatant was transferred to a clean tube.
6. 800 microliters of IgG beads was added and the tube was rotated for 2 hours.
7. Centrifuged at 3,000 rpm for 2 minutes and the supernatant was decanted.
8. The IgG beads were transferred and distributed to three parallel 10ml chromatography columns. Each column was washed with 10 ml washing buffer (WB) by applying 200 microliter aliquots. WB: 100 mM KCl, 10 mM HEPES pH 7.4, 5% glycerol, 1% DMSO, 0.1% TritonX100.
9. Washed with 400 microliters WB containing 2 mM DTT.
10. The beads were transferred with 2 ml WB containing 2 mM DTT to two eppendorf tubes and 500 units Tev protease was added. Rotated at 0° C. to 5° C. for 2 hours.
11. The beads and the buffer were transferred back to 10 ml chromatography column and the eluate was collected. The beads were washed with two more column volumes of WB containing 2 mM DTT and the eluates were combined in a 5 ml tube.
12. 500 microliters calmodulin beads and 5 mM CaCl₂ and 3 ml WBC (washing buffer for calmodulin beads were added. WBC—100 mM KCl, 10 mM HEPES pH 7.4, 5 mM DTT, 5 mM CaCl₂, 1 mM immidazol, 5% glycerol, 0.1% TritonX100. Rotated for 1 hour at 0° C. to 5° C.
13. The beads were transferred to 10 ml chromatography column and washed with 5 ml WBC.
14. The beads were transferred to a 5 ml tube and 3 ml WBC containing 0.3M KCl (WBC(0.3M)) was added. The tube was rotated for 10 minutes.
15. The beads and the buffer were transferred to a 10 ml chromatography column and the eluate was collected by gravity flow. The beads were washed with 2 column volumes of WBC (0.3M) and the eluates were combined.
16. The eluate was distributed to eppendorf tubes and ⅕ volume of 100% TCA was added.
17. Incubated on ice for 1 hour; centrifuged for 1 hour—14,000 rpm at 0° C.
18. The supernatant was decanted and 1 ml 90% acetone was added. Vortexed for 30 seconds and centrifuged for 10 minutes at 14,000 rpm. The supernatant was decanted.
19. Dried with a SpeedVac for 15 seconds.
20. Protein gel electrophoresis (SDS-PAGE—10% gel, 20 cm long) and silver staining were performed.

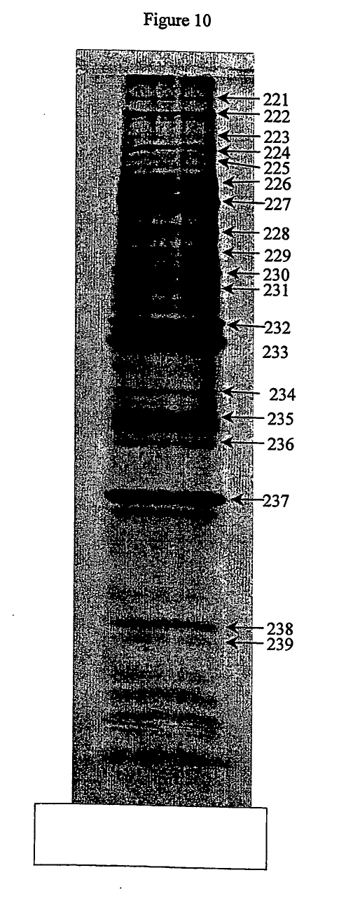

A gel image of high salt eluate from immobilized RNA Polymerase II from Saccharomyces cerevisiae is shown in FIG. 10. The carboxy-terminus of the chromosomal copy of the gene encoding Rpb1 is fused in-frame with a TAP-tag. 18 liters Saccharomyces cerevisiae culture was grown in YPD medium to OD600=0.7. The complexes were purified on IgG beads and further immobilized on calmodulin beads. The elution was performed with 0.5 M KCl in the WBC. A minor release of the fusion protein from the affinity matrix was observed.

Legend: 221—Rpb1, 222—Spt6, 223—Spt6, 224—Rpb2+Ctr9, 225—Ctr9+Spt6, 226—Tfg1+Prp22+Spt16+Spt6, 227—Tfg1+Fcp1+Spt16+Nip1, 228—Fcp1+Set2+Rtf1+Ygl244, 229—Tfg1+Fcp1+Ygr054+Leo1+Med1, 230—Pob3, 231—Paf1+Glo3, 232—Iws1+Tfg2, 233—TFg2, 234—Sua7(TFIIB homolog), 235—TFIIS, 236—Mpd2, 237—Tfg3, 238—Ess1, 239—Mbf1.

The following protocol was used:
1. The cells were collected by centrifugation at 4,000 rpm for 4 minutes and the medium was decanted. All subsequent manipulations were performed on ice or at 0° C. to 5° C.
2. The cells were suspended in 200 ml water and distributed into 50 ml plastic tubes; centrifuged at 4,000 rpm for 4 minutes and the water was decanted.
3. Frozen in liquid nitrogen for 1 minute.
4. The solid pellet was broken and protease inhibitors and 2-3 pieces (around 8 cm3) dry ice were added; ground with a coffee grinder for 1 minute; the powder was transferred to a beaker and, after allowing it to melt, the same volume of 2×Lysis buffer was added. Stirred with a plastic rod for 2 minutes. 2×Lysis Buffer: 200 mM KCl, 50 mM HEPES pH 7.4, 20% glycerol, 2% DMSO, 0.2% TritonX100, 2 mM PMSF, 2 mM benzamidine, 2×phosphatase inhibitors.
5. Centrifuged at 16,000 rpm for 30 minutes and the supernatant was transferred to a clean tube.
6. 800 microliters of IgG beads was added and the tube was rotated for 2 hours.
7. Centrifuged at 3,000 rpm for 2 minutes and the supernatant was decanted.
8. The IgG beads were transferred and distributed to three parallel 10 ml chromatography columns. Each column was washed with 10 ml washing buffer (WB) by applying 200 microliter aliquots. WB: 100 mM KCl, 10 mM HEPES pH 7.4, 5% glycerol, 1% DMSO, 0.1% TritonX100.
9. Washed with 400 microliters WB containing 2 mM DTT.
10. The beads were transferred with 2 ml WB containing 2 mM DTT to two eppendorf tubes and 500 units Tev protease was added. Rotated at 0° C. to 5° C. for 2 hours.
11. The beads were transferred back to 10 ml chromatography column and the eluates were collected. the beads were washed with two more column volumes of WB containing 2 mM DTT and the eluates were combined in a 5 ml tube.
12. 500 microliters calmodulin beads and 5 mM CaCl₂ and 3 ml WBC were added. WBC: 100 mM KCl, 10 mM HEPES pH 7.4, 5 mM DTT, 5 mM CaCl₂, 1 mM immidazol, 5% glycerol, 0.1% TritonX100. Rotated for 1 hr. The beads were transferred to 10 ml chromatography column and washed with 5 ml WBC.
13. The beads were transferred to a 5 ml tube and 3ml WBC containing 0.5M KCl (WBC (0.5)) was added. The tube was rotated for 10 minutes.
14. The beads and the buffer were transferred to a 10 ml chromatography column and the eluate was collected by gravity flow. The beads were washed with two column volumes of WBC(0.5M) and the eluates were combined.
15. The eluate was distributed to eppendorf tubes and ⅕ volume of 100% TCA was added.
16. Incubated on ice for 1 hour; centrifuged for 1 hour—14,000 rpm at 0° C.
17. The supernatant was decanted and 1 ml 90% acetone was added. Vortexed for 30 seconds and centrifuged for 10 minutes at 14,000 rpm. The supernatant was decanted.
18. Dried with a SpeedVac for 15 seconds.
19. Protein gel electrophoresis (SDS-PAGE—10% gel, 20 cm long) and silver staining were performed.

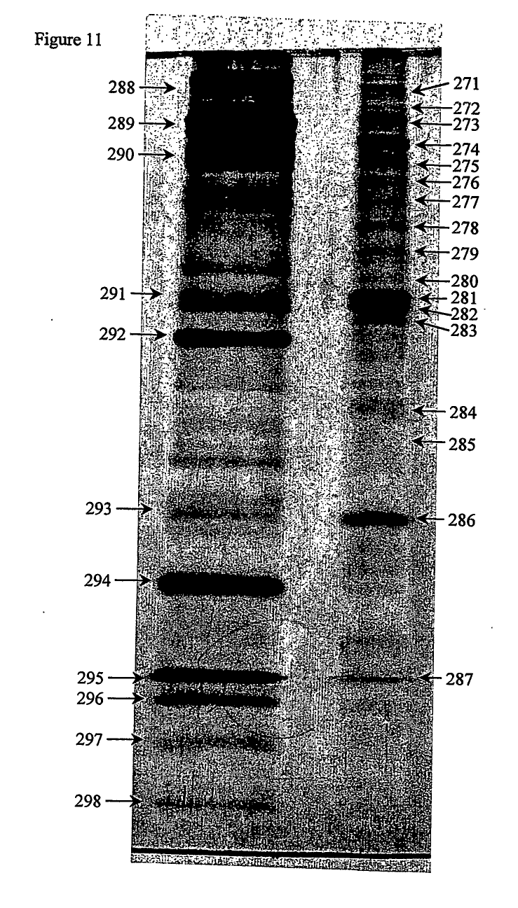

A gel image of high salt eluate from immobilized RNA Polymerase II from Saccharomyces cerevisiae and another eluate containing the core complex is shown in FIG. 11. The carboxy-terminus of the chromosomal copy of the gene encoding Rpb1 is fused in-frame with a TAP-tag. 9 liters Saccharomyces cerevisiae culture was grown in YPD medium to OD600=0.8. The protocol used for obtaining the high salt eluate shown in the right lane is the same as in FIG. 10 except for the high salt elution that was performed with 0.3 M KCl in the washing buffer. After separating the interacting proteins by high salt elution and thus completing the method of the invention, the immobilized RNA Polymerase II complexes were released by treatment with 5 mM EGTA and 0.5% SDS in WB. Half of the eluate was loaded in the left lane and subjected to PAGE-SDS in order to demonstrate the ratio between the amounts of the interacting proteins and the immobilized core complex. All gel bands, including the “blank” ones, were cut for analysis and the proteins that were identified with high confidence are indicated. Note the complete separation of the interacting proteins from the immobilized core complex by increasing the ionic strength (except TFIIF, which binds strongly to the core enzyme and was detected in both fractions). Different intensities of the bands in the two gel lanes illustrate the need to separate the substoichiometrically interacting proteins from the immobilized core complex. Note that a double gel well was used to load only a half of the SDS/EGTA eluate in order to avoid overloading and smearing of the gel.

Legend: 271—Spt6, 272—Ydl145 (Cop1), 273—Ctr9+Spt5, 274—Tfg1+Spt16+Lte1, 275—Tfg1+Spt5+Spt16+Fcp1, 276—Set2, 277—Fcp1+Leo1, 278—Pob3, 279—Paf1, 280—Paf1+Rtt103, 281—Tfg2+Iws1, 282—Tfg2, 283—Cdc73+Ceg1, 284—Dst1, 285—Rtt103, 286—Tfg3, 287—Ess1, 288—Rpb1, 289—Rpb2, 290—Tfg1, 291—Tfg2, 292—Rpb3, 293—Tfg3, 294—Rpb4, 295—Rpb5, 296—Rpb6, 297—Rpb7, 298—Rpb8.

A gel image of the high salt eluate from immobilized Rpb3 from Saccharomyces cerevisiae is shown in FIG. 12. The carboxyl-terminus of the chromosomal copy of the gene encoding Rpb3 is fused in-frame with a TAP-tag. The protocol used for obtaining the high salt eluate is the same protocol used in FIG. 10. Interacting proteins are indicated. In addition, trace amounts of the immobilized complex (Rpb1, Rpb2 and Rpb3) are shown.

The separation of the immobilized complex from the affinity matrix is not necessary for practicing the invention, it is outside the scope of the invention and, when practicing the invention, the greatest care must be taken not to separate the immobilized fusion protein from the affinity matrix during the separation of the interacting proteins from the immobilized fusion protein.

The contaminants that were determined by performing the method of the invention with two immobilizations on two different affinity matrices, i.e. IgG-sepharose and calmodulin beads, and with cellular lysate from organism gdevoid of affinity tagged protein, are: Tef2, Fks1, Fas2, Stm1, Rpl4, Tif34, Rpl5, Rps3, Rps1b, Rpl2, Prt1, Rpl8, Tef2, Rps2, Rps5, Rpl10, Ded1, Rpl9, Hsp60, Rps8, Gcd11, Rps20, Rpl20, Pab1, Rps17, Ssb1, Ssa2, Sse1, Rpl27, Rps14, Rps24, Mdn1, Tra1, Dyn1, Myo2, Ira2.

Although the usage of dual affinity tag results in low background from the residual contaminant proteins, the number of the detected interacting proteins is lower than the number of interacting proteins that are detected when a fusion protein with one tag (SpA-tag) is used. It is clear that the basic method of the invention (i.e. the protein of interest is fused to one affinity tag and only one immobilization of the protein of interest is performed before the separation of the interacting proteins) is faster, cheaper and results in identifying more interacting proteins. Compare the interacting proteins in FIG. 3, 4, 6, 7 and 8 with FIG. 9 and 10. Therefore, when the protein of interest is present in the cell in approximately 5,000-10,000 copies or higher, it is best if the basic method of the invention is carried out with a fusion protein containing one affinity tag. A good explanation for the loss of the interacting proteins when two sequential immobilization are performed is given by R. Aebersold and M. Mann, Nature, March 2003, Vol. 422. The background from the contaminant proteins creates problems mostly for the identification and analysis of the interacting proteins by mass spectrometry. If they are analyzed by other methods, e.g. immunoblofting, the background can be ignored. Also, the background from the contaminant proteins is negligible when compared to the background from the affinity tagged protein (or other subunits of the stable protein complex) that is encountered when a classical purification via affinity tag is performed.

The following proteins were identified by performing the method of the invention with several affinity tagged subunits of RNA Polymerase II from Saccharomyces cerevisiae. Proteins separated by a slash are subunits of stable protein complexes.
(a) transcription factors that are known to bind to RNA Polymerase II and proteins associated with them: Tfg1/Tfg2/Tfg3—TFIIF, Spt5, TFIIS, Spt6/Iws1, Fcp1, Cet1/Ceg1, TFIIB, Rgr1/Srb4/Med1/Med4—mediator subunits, Ppn1—interact with TFIIB, Nip1—interact with TFIIB, Rtf1/Paf1/Ctr9/Leo1/Cdc73—PAF complex, Spt16/Pob3, Taf7/Taf12—TFIID, Ess1, Rad3/Tfb2—TFIIH, Tfa2 (TFIIE).
(b) Proteins that are known to be involved in mRNA transcription or mRNA processing but are not known to associate directly or indirectly with the core enzyme: Npl3—DNA binding; involved in mRNA transport, Hrp1—mRNA cleavage/polyadenylation factor, Prp43—mRNA splicing factor, Syf1—mRNA splicing factor, Smb1—mRNA splicing factor, Puf2—mRNA binding protein, Gcd14—RNA processing/modification factor, Fip1—mRNA polyadenylation factor, Clp1—cleavage-polyadenylation factor IA subunit, Ylr419—RNA helicase; involved in RNA splicing, Rgm1—transcription factor, Rgt1—transcriptional activator, Cdc39—transcription factor, Ygl244—transcription cofactor activity, Mbf1—bridges Gcn4and Spt15, Stb5—transcriptional activator,
(c) Proteins that interact with DNA/chromatin and/or mRNA and/or NTPs: Rap1—interacts with Rif1, DNA-binding protein, Rif2—interacts with Rap1 and with Rif1, Ddr48—DNA damage inducible protein, Ddc1—involved in DNA damage checkpoint, Prp22—helicase-like protein, Prp18—helicase-like protein, Cdc6—GTPase/ATPase; cell cycle control, Ygr054—part of Swi/Snf and Npl3 complexes, Sdc25—GDP/GTP exchange factor for Ras, Glo3—GTPase activator,
(d) other proteins: Sap4—associates with the SIT4 phosphatase, Sap185—SIT4 associated protein, Ptc2—protein phosphatase type 2C, Ubp8—putative deubiquitinating enzyme, Kin4—nuclear protein kinase, Hbt1—Hub1 target, Yhr121w—Stb5 associated, Sup35—interacts with Mip6 and Nab3, Sip1—Snf1 protein kinase substrate, Rtt103—binds to CTD, Mks1, Ydl145 (Cop1), Mpd2—protein disulfide isomerase. More proteins are indicated in the Examples.

The nucleotide sequences of the genes and amino acid sequences of the proteins can be found by submitting the ORF name or the name of the protein at: Saccharomyces Genome Database at yeastgenome.org/ or, at National Center for Biotechnology Information web site at ncbi. nlm. nih.gov:80/mapview/map_search.cgi?taxid=4932.

Note that by using only one method, i.e. the method of the invention, it is possible to identify directly nearly all proteins that are known to interact with RNA Polymerase II and that were identified during the last 20 years by using various methods (including genetic based and library based methods).

Note that among the detected proteins are two well-known enzymes that modify covalently the carboxyl-terminal domain (CTD) of RNA Polymerase II: (a) Fcp1—a TFIIF interacting phosphatase that recycles RNA Polymerase II, (b) Ess1—a prolyl isomerase of the CTD. Since post-translational modifications play a major role in modulating the protein function, the identification of the modifying enzyme for a protein of interest is an important application of the invention. In addition, the invention can be used to identify the substrate for an enzyme of interest.

The presence of many (more than 30) well-established transcription factors in the high salt eluates obtained from immobilized RNA Polymerase II is enough for validation of the invention, i.e. the proteins isolated by the method of the invention are true interacting proteins. One way to validate interacting proteins is to perform the method of the invention with a fusion protein containing the putative interacting protein. The presence of the first protein of interest among the interacting proteins increases the confidence that the protein-protein interaction is physiologically relevant. For example, by performing the method of the invention with tagged subunits of RNA Polymerase II, the three subunits of the transcription factor TFIIF (Tfg1, Tfg2 and Tfg3) are detected in the high salt eluate, and, on the other hand, by performing the method of the invention with tagged Tfg1, the subunits of the RNA Polymerase II core enzyme are detected (FIGS. 7 and 8).

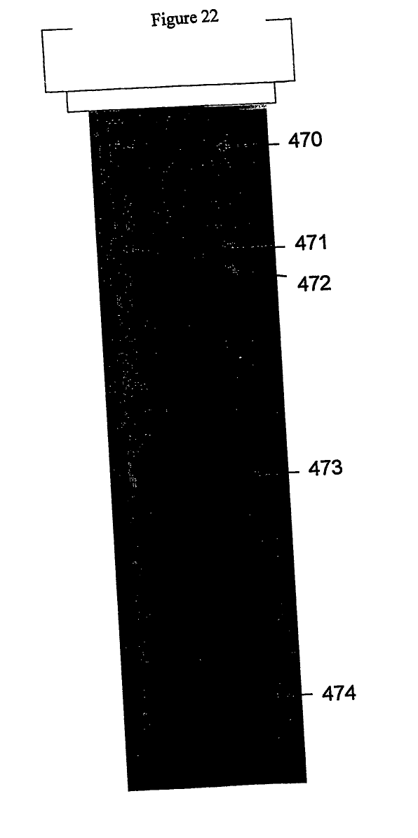

Alternatively, the validation can be performed by other methods for detection of protein interactions as described by Phizicky E. and Fields S. in Microbiological Reviews, Mar. 1995, Vol. 95, No. 1, or as described by Phizicky E, Bastiaens PI, Zhu H, Snyder M, Fields S., Nature 2003. For example, purification via an affinity tag fused to the putative interactor can be performed. Rtt103 is among the interacting proteins detected by the method of the invention and, in order to validate it as an interactor, a tandem affinity purification was performed with a strain expressing TAP-tagged Rtt103. As shown in FIG. 22, two subunits of the RNA Polymerase II core complex (Rpb1 and Rpb2) co-purified with Rft103. Legend: 470—Rpb1, 471—Rpb2, 472—Rat1, 473—Rtt103, 474—Rai1.

### Example 5

### Drug Screening and Drug Discovery

The invention can be used for monitoring the effect of a pre-drug on a protein-protein interaction that is associated with a disease. The protein can be fused to a SpA-tag but if the cellular lysate or other biological fluid contains a high level of immunoglobulins or other proteins that bind to SpA-tag or IgG, a different affinity tag should be used (e.g. MBP or SBP or GST). In this example, the protein does not contain a pathogenic mutation. It is best if a complete proteomic map has been built around the protein of interest before the experiment, so that the presence of interacting proteins in the eluate can be determined by immunoassays or other sensitive methods.

Cellular lysate or a fraction of cellular lysate or other biological fluid from human cells containing a modified gene that encodes a fusion protein containing the SpA-tag is mixed with IgG-beads. After removing the unbound substances, the immobilized complex is treated with washing buffer containing the pre-drug in concentration between 0 and 1 mM. The concentration of the pre-drug can vary. The effect of the pre-drug is determined by monitoring the presence of interacting proteins in the eluate. The following protocol can be used (all manipulations are performed at 0° C.-5° C):
1. The cells are collected and frozen in liquid nitrogen for 1 minute. After melting and adding lysis buffer, the lysate is homogenized by 10-20 strokes. Cellular lysate from 5×10⁹ mammalian cells in 10 ml Lysis Buffer (50 mM KCl, 10 mM HEPES pH 7.4, 10% glycerol, 1% DMSO, 0.1% TritonX100, 0.2 mM PMSF, 0.2 mM benzamidine,) is centrifuged at 16,000 rpm for 20 minutes and the supernatant is transferred to a clean tube.
2. Adding 100 microliters of IgG beads and rotating the tube for 1.5 hours.
3. Centrifugation at 3,000 rpm for 2 minutes and decanting the supernatant.
4. Transferring the beads to a 10 ml chromatography column. Washing the column with 10 ml washing buffer (WB) by applying 200 microliter aliquots (WB—50 mM KCl, 10 mM HEPES pH 7.4, 5% glycerol, 0.2% DMSO, 0.1% TritonX100, 0.2 mM PMSF, 0.2 mM benzamidine).
5. Transferring the beads to a 2 ml tube and adding 0.5 ml WB containing 1 mM pre-drug.
6. Rotating the tube for 30 minutes.
7. Transferring the beads and the buffer to a 10 ml chromatography column and collecting the eluate by gravity flow. Washing the beads with two column volumes of WB containing 1 mM pre-drug and combining the eluates.
8. Analyzing the eluate for the presence or absence of interacting proteins or other biomolecules in order to determine the effect of the pre-drug on the complex.

The above method can be modified in several ways:
(I) The fusion protein contains a pathogenic mutation and the pre-drug is present in all solutions: growing medium, lysis buffer and washing buffer. In steps 5, 6 and 7, the elution is performed by WB without a pre-drug in order to check whether the absence of the pre-drug leads to dissociation of the complex.
(II) Another variant is to perform the treatment in steps 5, 6 and 7 with WB containing 0.3M KCl and a pre-drug. Presumably, the concentration of the drug should be lower when it is applied to living cells (i.e. in vivo) and higher when it is applied to the buffers (i.e. in vitro).

If the pre-drug affects the complex at 1 mM concentration, the experiment can be repeated with lower concentrations of the pre-drug, e.g. 100 mcM or 10 mcM or 1 mcM, in order to determine the lowest concentration that affects the complex. After conducting several experiments, the pre-drug can be modified and experiments can be repeated in order to determine whether the modification increases the specificity of the pre-drug, i.e. whether the pre-drug affects the complex at lower concentrations.

In another example, before conducting the drug treatment experiments, a proteomic map can be built around the protein of interest so that it is known which protein-protein interaction is associated with a particular disease. In this case, both proteins can be tagged with different tags and the interacting protein can be detected by immunoassays or other assays in order to reduce the scale of the experiment. Columns containing as low as 10 mcl or 20 mcl affinity matrix can be used (Formosa T., Methods Enzymol. 1991). Alternatively, if both proteins are present in a very low copy number in the cell, they can be over-expressed as fusion proteins in an exogenous or endogenous organism and the experiment can be performed with purified proteins. In a further example, the proteins can be over-expressed in their endogenous organism and the regular method of the invention can be carried out. Again, the over-expression should not lead to formation of inclusion bodies.

Two types of control experiments can be performed in order to validate the effect of the pre-drug:
(I) step 5, 6 and 7 are performed without a pre-drug; or
(II) testing the effect of a pre-drug on one or more biologically unrelated protein complexes.

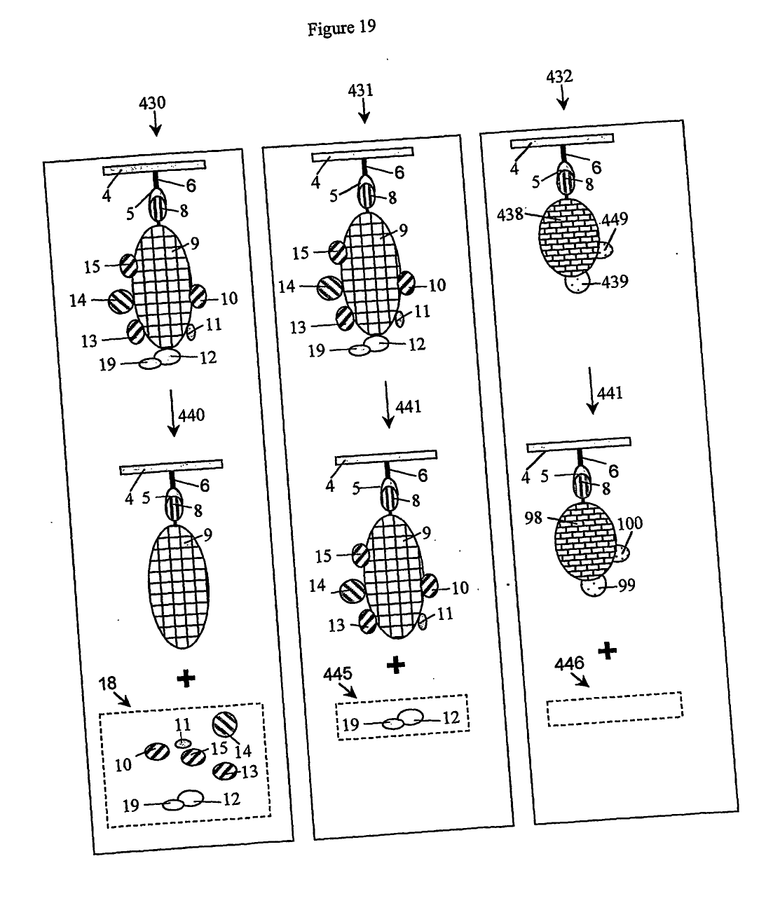

FIG. 19 illustrates the selective effect of a drug on a particular protein-protein interaction.

Legend: 430—left panel: the immobilized protein of interest is treated with nonspecific elution agent and all interacting proteins are separated, 431—middle panel: the immobilized protein of interest is treated with specific elution agent which causes separation of only one interacting protein complex, 432—right panel: the unrelated protein complex is treated with the same specific elution agent but there is no separation of the interacting proteins, 438—unrelated protein, 439—protein interacting with the unrelated protein, 449—another protein interacting with the unrelated protein, 440—elution with nonselective elution agent (e.g. increased salt concentration), 441—elution with selective agent (e.g. drug treatment), 18—eluate obtained by the method of the invention, i.e. proteins eluted by treatment with a nonselective elution agent, 445—proteins eluted by treatment with a selective agent; 446—the unrelated protein complex is not affected by the treatment with a selective agent and the interacting proteins are not eluted.

### Example 6

### Benefits and Features of the Method of the Invention

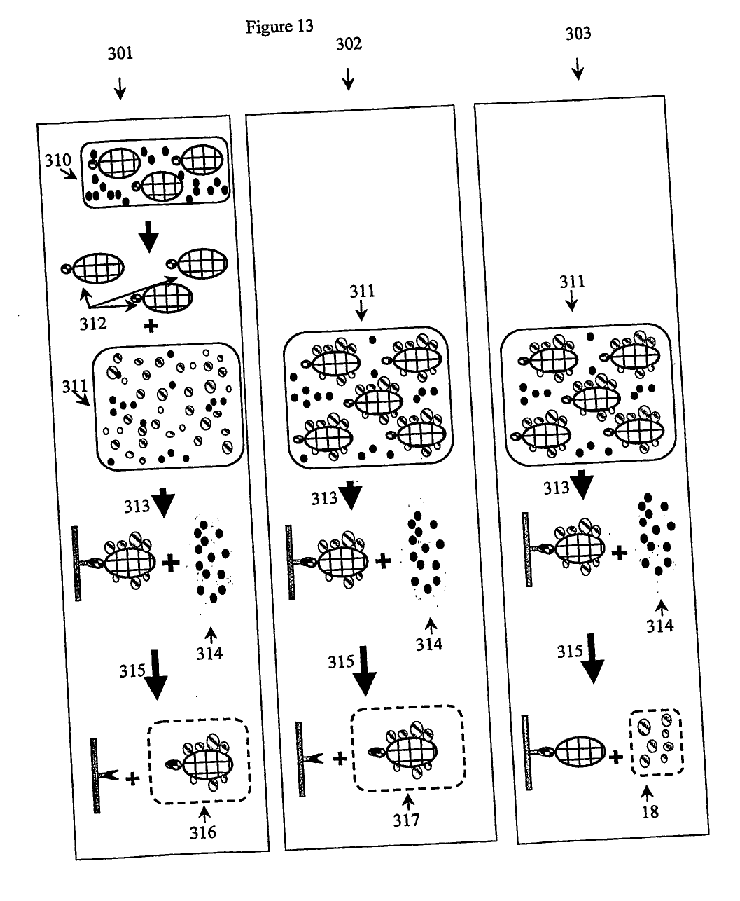

FIG. 13 shows a comparison between the method of the invention and other methods for detecting protein-protein interactions that utilize affinity tagged proteins.

Legend: 301—the left panel illustrates GST pull down, 302—the middle panel illustrates purification via an affinity tag, 303—the right panel illustrates the method of the invention. 310—exogenous organism (e.g. Escherichia coli), 311—endogenous organism, 312—affinity tagged protein, 313—the affinity tagged protein and the interacting proteins are immobilized on solid phase, 314—unbound proteins and/or other substances (solid black) are removed, 315—isolating the interacting proteins—only in the case of the invention they are separated from the affinity tagged protein (the eluate does not contain affinity tagged protein), 316—eluate obtained by GST pull down; 317—eluate obtained by purification via an affinity tag; 18—eluate obtained by the method of the invention.

GST pull down—a single fusion protein is expressed in an exogenous organism (most often E. coli) and is used to prepare the affinity column. GST pull down can be performed by separating the fusion protein from the affinity matrix or by separating the interacting proteins from the immobilized protein of interest.

Purification via an affinity tag—the fusion protein and the interacting proteins are isolated together. Only in rare cases can transient complexes be detected.

The invention—the substoichiometrically interacting proteins are separated from the immobilized fusion protein and the dynamic range problem and/or other problems are eliminated.

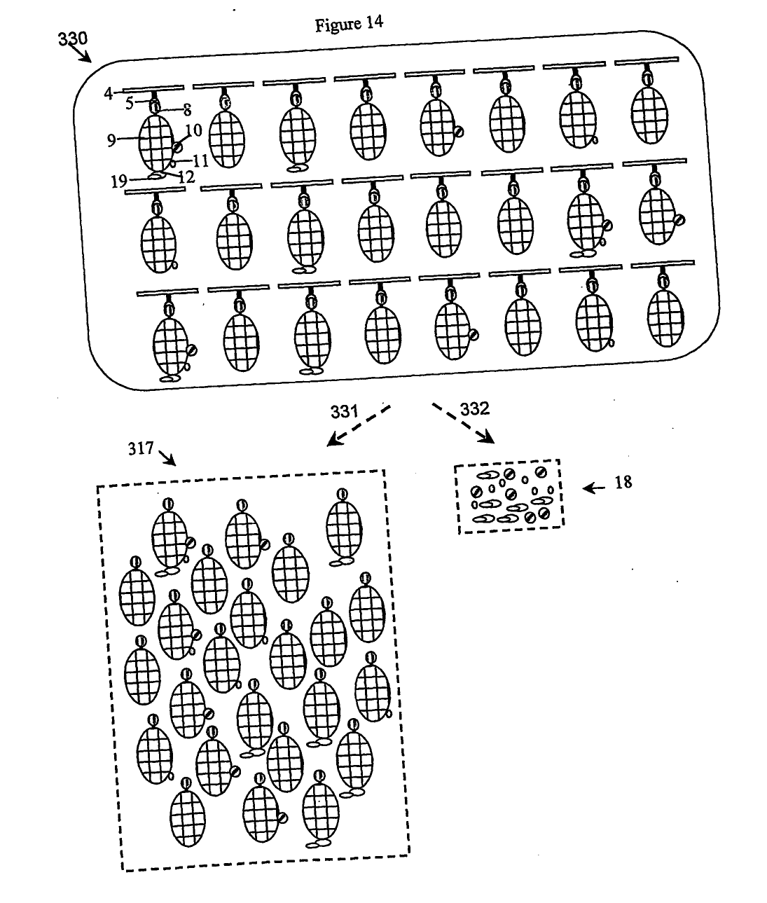

FIG. 14 illustrates the necessity to separate substoichiometrically interacting proteins from the high abundance fusion protein. The bulk of the fusion protein is immobilized via an affinity tag whereas only the fraction of interacting proteins that has been bound to the fusion protein in vivo is immobilized.

Legend: 330—transient complexes are immobilized on an affinity matrix via an affinity tag, 331—purification via an affinity tag, 332—purification by the method of the invention, 317—eluate obtained by purification via an affinity tag, 18—eluate obtained by the method of the invention.

Note the amount of the tagged protein that is not associated with interacting proteins. In the illustration the ratio between the fusion protein and each interacting protein is 4:1 but in reality, the ratio can vary between 10:1 and more than 100:1. Moreover, when the fusion protein is a subunit of a stable protein complex, the problems arising from the different stoichiometries are even more complicated.

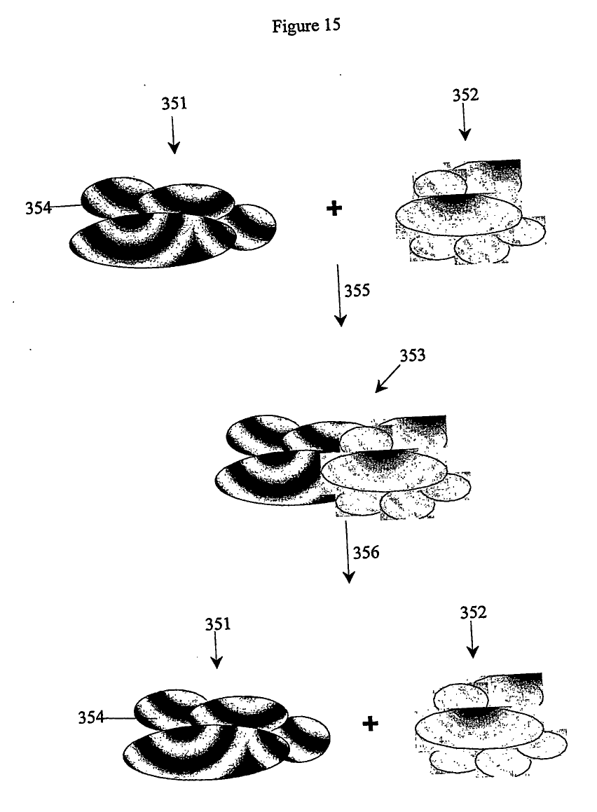

FIG. 15 illustrates the formation and disintegration of a transient protein complex between two stable (permanent) protein complexes (first and second vertical arrows respectively).

Legend: 351—stable protein complex with 4 subunits, 352—stable protein complex with 6 subunits, 353—transient protein complex between the two stable complexes, 354—subunit of a stable complex that binds indirectly to another stable complex, 355—formation of a transient protein complex, 356—disintegration of the complex.

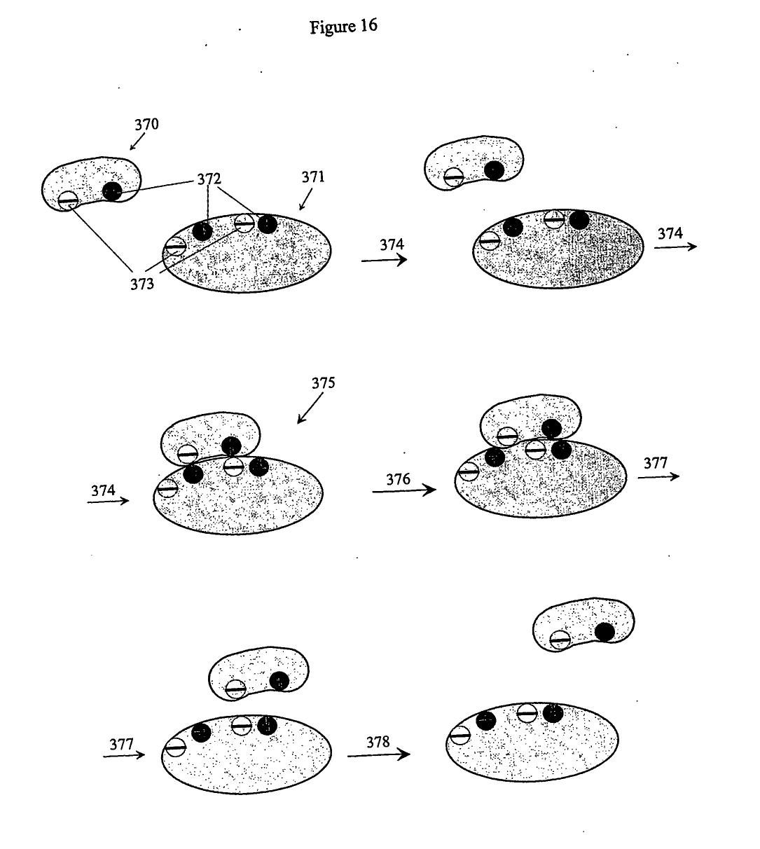

FIG. 16. A model for transient protein-protein interactions according to which formation and disintegration of transient protein complexes is due to alternation of electrostatic attraction and electrostatic repulsion between the electrostatically charged amino acids.

Legend: 370—first interacting protein, 371—second interacting protein, 372—positively charged amino acids, 373—negatively charged amino acids, 374—electrostatic attraction, 375—two interacting proteins form a complex, 376—change of position or orientation of the two interacting proteins relative to one another, 377—electrostatic repulsion, 378—transient complex disintegrates.

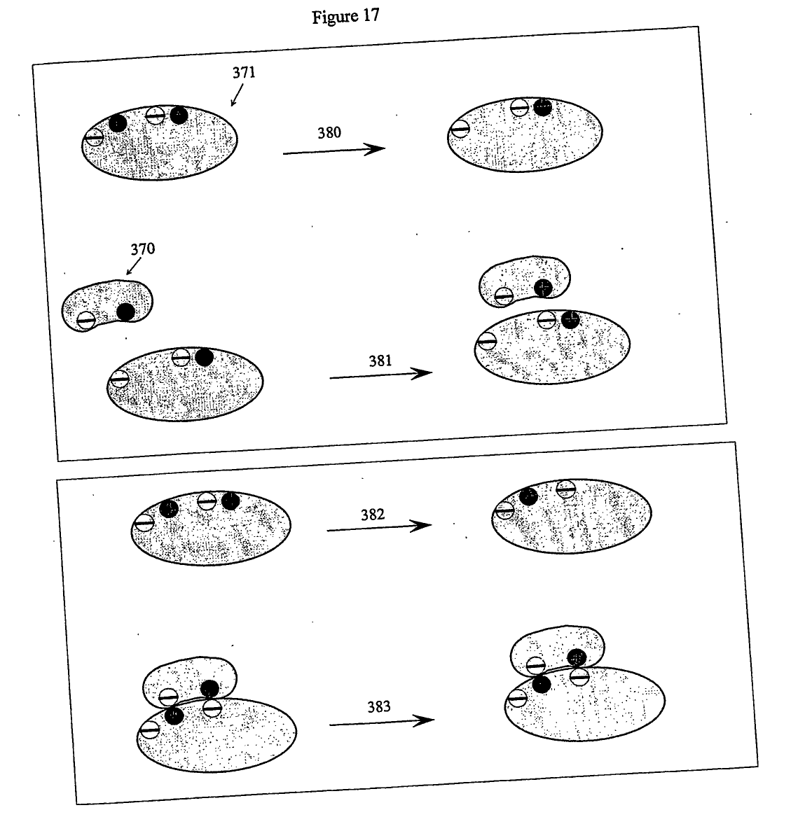

FIG. 17. When a mutation changes the electrostatic properties of the protein, the transient interaction cannot occur properly.

Legend for the upper panel: 380—mutation leading to disappearance of positively charged amino acid, 381—electrostatic attraction is prevented. Legend for the lower panel: 382—mutation leading to disappearance of different positively charged amino acid, 383—electrostatic repulsion is prevented and the interacting proteins remain stuck together.

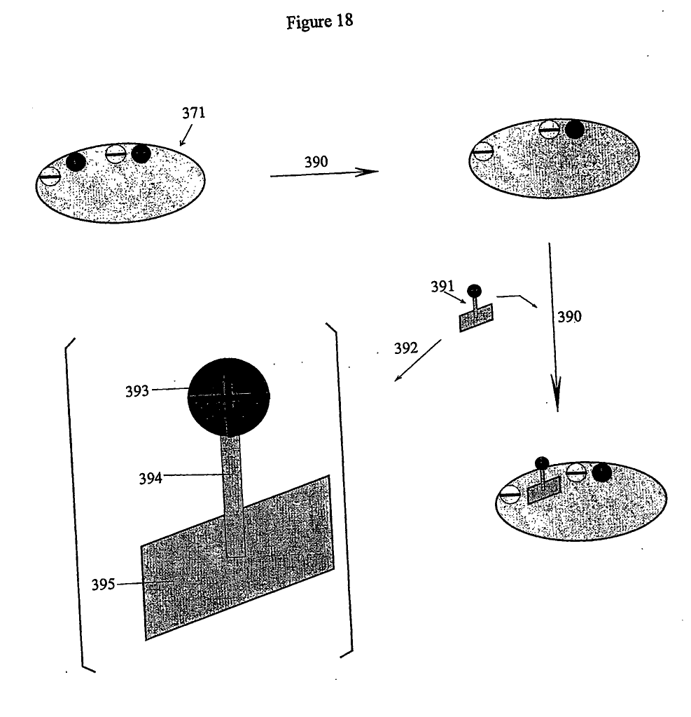

FIG. 18. Method for drug design and schematic structure of a chemical or a biomolecule that binds to a mutant protein and restores (at least partially) its electrostatic properties. As a result, the protein is capable of forming a protein complex.

Legend: 390—drug treatment, 391—a drug, 392—an exploded view of the drug, 393—part that mimics and/or restores the original charge, 394—linker (optional), 395—part that binds selectively to the mutant protein.

When the disease is caused by a mutation that converts a permanent protein-protein association into a weak interaction, the drug should stabilize the protein-protein association. Drugs that are designed or discovered according to the invention, can be defined as chemicals that restore the equilibrium binding constant of a mutant protein complex back to that of the respective native one.

If a human disease has an analog in another organism (e.g. mouse or rat), a heterologous nucleic acid can be administered into the organism so that it expresses an affinity tagged protein of interest. In this case, the method of the invention can be performed with cellular lysate or other biological fluids from the sacrificed organism.

### Example 7

### Variations of the Method with Respect to the Affinity Tag

The following examples illustrate that the methods of the invention can be carried out with different affinity tags. The scale of the experiments is suitable for detection by mass spectrometry of proteins interacting with a protein of interest that is present in approximately 5,000-10,000 copies per cell or more.

If a heterologous nucleic acid is introduced into a Saccharomyces cerevisiae strain such that the organism can express a fusion protein containing a GST-tag₁ then the following protocol can be used:
1. Growing 12 liters Saccharomyces cerevisiae culture in YPD medium to OD600=0.8.
2. Collecting the cells by centrifugation at 4,000 rpm for 4 minutes and decanting the medium. All subsequent manipulations are performed on ice or at 0° C. to 5° C.
3. Suspending the cells in 100 ml water and distributing into 50 ml plastic tubes; centrifugation at 4,000 rpm for 4 minutes and decanting the water.
4. Freezing in liquid nitrogen for 1 minute.
5. Breaking the solid pellet and adding protease inhibitors and 2-3 pieces (around 8 cm³) of dry ice; grinding with a coffee grinder for 1 minute; transferring the powder to a beaker and, after allowing it to melt, adding the same volume of 2×Lysis buffer. Stirring with a plastic rod for 2 minutes. 2×Lysis Buffer: 100 mM KCl, 50 mM HEPES pH 7.4, 20% glycerol, 2% DMSO, 0.2% TritonX100, 2 mM PMSF, 2 mM benzamidine.
6. Centrifugation at 16,000 rpm for 30 minutes and transferring the supernatant to a clean tube.
7. Adding 500 microliters of glutathione beads and rotating the tube for 2 hours.
8. Centrifugation at 3,000 rpm for 2 minutes and decanting the supernatant.
9. Transferring and distributing the glutathione beads to two parallel 10 ml chromatography columns. Washing each column with 10 ml washing buffer (WB) by applying 200 microliter aliquots. WB: 50 mM KCl, 10 mM HEPES pH 7.4, 5% glycerol, 1% DMSO, 0.1% TritonX100.
10. Transferring the beads to a 5 ml tube and adding 4 ml WB with 0.4M KCl (WB (0.4M)).
11. Rotating the tube for 10 minutes.
12. Transferring the beads and the buffer to a 10 ml chromatography column and collecting the eluate by gravity flow. Washing the beads with two column volumes of WB(0.4M) and combining the eluates.
13. Analyzing the interacting proteins and/or other biomolecules in the eluate. Optionally, proceed to 14.
14. Distributing the eluate to eppendorf tubes and adding ⅕ volume of 100% TCA.
15. Incubating on ice for 30 minutes; centrifugation for 30 minutes—14,000 Rpm at 0° C. to 5° C.
16. Decanting the supernatant and adding 1 ml 90% acetone. Vortexing for 30 seconds and centrifuging for 10 minutes at 14,000 rpm. Decanting the supernatant.
17. Drying with SpeedVac for 15 seconds.
18. Protein gel electrophoresis (SDS-PAGE—10% gel, 20 cm long) and silver staining.

When the interacting proteins are isolated in low amounts and an additional affinity purification step is needed before the separation from the immobilized fusion protein, a variety of combinations of two affinity tags can be used. For example, the combination can be a GST-tag and a SpA-tag. The following protocol can be used in several cases:
(a) One subunit of protein complex is fused to a GST-tag and another subunit is fused to a SpA-tag,
(b) The protein or subunit of the protein complex contains a GST-tag at the N-terminus and a SpA-tag at the C-terminus,
(c) Both tags can be located at the same end.
1. Growing 18 liters Saccharomyces cerevisiae culture in YPD medium to OD600=0.8.
2. Collecting the cells by centrifugation at 4,000 rpm for 4 minutes and decanting the medium. All subsequent manipulations are performed on ice or at 0° C. to 5° C.
3. Suspending the cells in 200 ml water and distributing into 50 ml plastic tubes; centrifugation at 4,000 rpm for 4 minutes and decanting the water.
4. Freezing in liquid nitrogen for 1 minute.
5. Breaking the solid pellet and adding protease inhibitors and 2-3 pieces (around 8 cm³) of dry ice; grinding with a coffee grinder for 1 minute; transferring the powder to a beaker and, after allowing it to melt, adding the same volume of 2×Lysis buffer. Stirring with a plastic rod for 2 minutes. 2×Lysis Buffer: 200 mM KCl, 50 mM HEPES pH 7.4, 20% glycerol, 2% DMSO, 0.2% TritonX100, 2 mM PMSF, 2 mM benzamidine.
6. Centrifugation at 16,000 rpm for 30 minutes and transferring the supernatant to a clean tube.
7. Adding 800 microliters of glutathione beads and rotating the tube for 2 hours.
8. Centrifugation at 3,000 rpm for 2 minutes and decanting the supernatant.
9. Transferring and distributing the glutathione beads to four parallel 10 ml chromatography columns. Washing each column with 10 ml washing buffer (WB) by applying 200 microliter aliquots. WB: 100 mM KCl, 10 mM HEPES pH 7.4, 5% glycerol, 1% DMSO, 0.1% TritonX100.
10. Transferring the beads to a 5 ml tube and adding 3 ml WB containing 10 mM reduced glutathione.
11. Rotating the tube for 10 minutes.
12. Transferring the beads and the buffer to a 10 ml chromatography column and collecting the eluate by gravity flow. Washing the beads with two column volumes of WB containing 10 mM reduced glutathione and combining the eluates in a 5 ml tube.
13. Adding 600 microliters of IgG beads to the eluate and rotating the tube for 1 hour.
14. Centrifugation at 3,000 rpm for 2 minutes and decanting the supernatant.
15. Transferring and distributing the IgG beads to two parallel 10 ml chromatography columns. Washing each column with 10 ml WB by applying 200 microliter aliquots.
16. Transferring the beads to a 5 ml tube and adding 3 ml WB containing 0.5M KCl (WB(0.5M)). Rotating the tube for 10 minutes.
17. Transferring the beads and the buffer to a 10 ml chromatography column and collecting the eluate by gravity flow. Washing the beads with two column volumes of WB(0.5M) and combining the eluates.
18. Analyzing the eluate.

Another example is a combination of a SpG-tag and a SBP (Streptavidin binding peptide). The following protocol can be used:
1. Growing 12 liters Saccharomyces cerevisiae culture in YPD medium to OD600=0.9.
2. Collecting the cells by centrifugation at 4,000 rpm for 4 minutes and decanting the medium. All subsequent manipulations are performed on ice or at 0° C. to 5° C.
3. Suspending the cells in 200 ml water and distributing into 50 ml plastic tubes, centrifugation at 4,000 rpm for 4 minutes and decanting the water.
4. Freezing in liquid nitrogen for 1 minute.
5. Breaking the solid pellet and adding protease inhibitors and 2-3 pieces (around 8 cm³) of dry ice; grinding with a coffee grinder for 1 minute; transferring the powder to a beaker and, after allowing it to melt, adding the same volume 2×Lysis buffer. Stirring with a plastic rod for 2 minutes. 2×Lysis Buffer: 200 mM KCl, 50 mM HEPES pH 7.4, 20% glycerol, 2% DMSO, 0.2% TritonX100, 2 mM PMSF, 2 mM benzamidine.
6. Centrifugation at 16,000 rpm for 20 minutes and transferring the supernatant to a clean tube.
7. Adding 600 microliters streptavidin beads and rotating the tube for 1.5 hours.
8. Centrifugation at 3,000 rpm for 2 minutes and decanting the supernatant.
9. Transferring and distributing the streptavidin beads to three parallel 10 ml chromatography columns. Washing each column with 10 ml washing buffer (WB) by applying 200 microliter aliquots. WB: 100 mM KCl, 10 mM HEPES pH 7.4, 5% glycerol, 1% DMSO, 0.1% TritonX100.
10. Transferring the beads to a 5 ml tube and adding 4 ml WB containing 4 mM biotin.
11. Rotating the tube for 10 minutes.
12. Transferring the beads and the buffer to 10 ml chromatography column and collecting the eluate by gravity flow. Washing the beads with two column volumes of WB containing 4 mM biotin and combining the eluates in a 5 ml tube.
13. Adding 600 microliters of IgG beads to the eluate and rotating the tube for 1 hour.
14. Centrifugation at 3,000 rpm for 2 minutes and decanting the supernatant.
15. Transferring and distributing the IgG beads to three parallel 10 ml chromatography columns. Washing each column with 10 ml WB by applying 200 microliter aliquots.
16. Transferring the beads to a 5 ml tube and adding 3.5 ml WB containing 0.5M KCl (WB(0.5M)). Rotating the tube for 10 minutes.
17. Transferring the beads and the buffer to a 10 ml chromatography column and collecting the eluate by gravity flow. Washing the beads with two column volumes of WB(0.5M) and combining the eluates.
18. Analyzing the eluate.

Another example is a combination of a MBP and a HA-tag. The following protocol can be used:
1. Growing 12 liters Saccharomyces cerevisiae culture in YPD medium to OD600=0.9.
2. Collecting the cells by centrifugation at 4,000 rpm for 4 minutes and decanting the medium. All subsequent manipulations are performed on ice or at 0° C. to 5° C.
3. Suspending the cells in 200 ml water and distributing into 50 ml plastic tubes, centrifugation at 4,000 rpm for 4 minutes and decanting the water.
4. Freezing in liquid nitrogen for 1 minute.
5. Breaking the solid pellet and adding protease inhibitors and 2-3 pieces (around 8 cm³) of dry ice; grinding with a coffee grinder for 1 minute; transferring the powder to a beaker and, after allowing it to melt, adding the same volume 2×Lysis buffer. Stirring with a plastic rod for 2 minutes. 2×Lysis Buffer: 200 mM KCl, 50 mM HEPES pH 7.4, 20% glycerol, 2% DMSO, 0.2% TritonX100, 2 mM PMSF, 2 mM benzamidine.
6. Centrifugation at 16,000 rpm for 20 minutes and transferring the supernatant to a clean tube.
7. Adding 800 microliters of anti-HA affinity matrix and rotating the tube for 2 hours.
8. Centrifugation at 3,000 rpm for 2 minutes and decanting the supernatant.
9. Transferring and distributing the anti-HA affinity matrix to four parallel 10 ml chromatography columns. Washing each column with 10 ml washing buffer (WB) by applying 200 microliter aliquots. WB: 100 mM KCl, 10 mM HEPES pH 7.4, 5% glycerol, 1% DMSO, 0.1% TritonX100.
10. Transferring the beads to a 5 ml tube and adding 3 ml WB containing HA-tag at 1 mg/ml.
11. Rotating the tube for 10 minutes.
12. Transferring the beads and the buffer to 10 ml chromatography column and collecting the eluate by gravity flow. Washing the beads with two column volumes of WB containing HA peptide at 1 mg/ml and combining the eluates in a 10 ml tube.
13. Adding 600 microliters of cross-linked amylose beads to the eluate and rotating the tube for 1 hour.
14. Centrifugation at 3,000 rpm for 2 minutes and decanting the supernatant.
15. Transferring and distributing the beads to three parallel 10 ml chromatography columns. Washing each column with 10 ml WB by applying 200 microliter aliquots.
16. Transferring the beads to a 5 ml tube and adding 3.5 ml WB containing 0.5M KCl (WB(0.5M)). Rotating the tube for 10 minutes.
17. Transferring the beads and the buffer to a 10 ml chromatography column and collecting the eluate by gravity flow. Washing the beads with two column volumes of WB(0.5M) and combining the eluates.
18. Analyzing the eluate.

Another example is a combination of a SBP (Streptavidin binding peptide) and a poly-His tag.

The following protocol can be used:

1. Growing 18 liters Saccharomyces cerevisiae culture in YPD medium to OD600=0.7.
2. Collecting the cells by centrifugation at 4,000 rpm for 4 minutes and decanting the medium. All subsequent manipulations are performed on ice or at 0° C. to 5° C.
3. Suspending the cells in 200 ml water and distributing into 50 ml plastic tubes; centrifugation at 4,000 rpm for 4 minutes and decanting the water.
4. Freezing in liquid nitrogen for 1 minute.
5. Breaking the solid pellet and adding protease inhibitors and 2-3 pieces (around 8 cm³) of dry ice; grinding with a coffee grinder for 1 minute; transferring the powder to a beaker and, after allowing it to melt, adding the same volume of 2×Lysis buffer. Stirring with a plastic rod for 2 minutes. 2×Lysis Buffer: 200 mM KCl, 50 mM HEPES pH 7.4, 20% glycerol, 2% DMSO, 0.2% TritonX100, 2 mM PMSF, 2 mM benzamidine.
6. Centrifugation at 16,000 rpm for 20 minutes and transferring the supernatant to a clean tube.
7. Adding 800 microliters streptavidin beads and rotating the tube for 1.5 hours.
8. Centrifugation at 3,000 rpm for 2 minutes and decanting the supernatant.
9. Transferring and distributing the streptavidin beads to four parallel 10 ml chromatography columns. Washing each column with 10 ml washing buffer (WB) by applying 200 microliter aliquots. WB: 100 mM KCl, 10 mM HEPES pH 7.4, 5% glycerol, 1% DMSO, 0.1% TritonX100.
10. Transferring the beads to a 5 ml tube and adding 4 ml WB containing 4 mM biotin.
11. Rotating the tube for 10 minutes.
12. Transferring the beads and the buffer to 10 ml chromatography column and collecting the eluate by gravity flow. Washing the beads with two column volumes of WB containing 4 mM biotin and combining the eluates in a 10 ml tube.
13. Adding 600 microliters of Ni2+-NTA resin to the eluate and rotating the tube for 1 hour.
14. Centrifugation at 3,000 rpm for 2 minutes and decanting the supematant.
15. Transferring and distributing the Ni2+-NTA resin to three parallel 10 ml chromatography columns. Washing each column with 10 ml WB by applying 200 microliter aliquots.
16. Transferring the Ni2+-NTA resin to a 5 ml tube and adding 3.5 ml WB containing 0.4M KCl (WB(0.4M)). Rotating the tube for 10 minutes.
17. Transferring the beads and the buffer to a 10 ml chromatography column and collecting the eluate by gravity flow. Washing the beads with two column volumes of WB(0.4M) and combining the eluates.
18. Analyzing the eluate.

When TAP-tag is used and the first immobilization is on calmodulin beads, the lysis buffer and washing buffer should contain 2 mM Ca++. After removing the unbound substances and releasing the fusion protein by 2 mM EGTA treatment, the fusion protein is immobilized on IgG beads and the residual unbound substances are removed. The separation of the interacting proteins and/or other substances from the immobilized fusion protein is performed with WB containing 0.6 M KCl.

Examples of carrying out the invention with a protein of interest devoid of an affinity tag:

A specific antibody can be raised against the protein of interest and subsequently immobilized on a solid support. Cellular lysate or a fraction of cellular lysate from an organism lacking an affinity tagged protein is mixed with antibody coated solid support and after removing the unbound substances, biomolecules that are bound to the protein of interest are separated from it by increasing the salt concentration to 0.3M KCl. The following protocol can be used (all manipulations are performed at 0° C. to 5° C):
1. The cells are collected and frozen in liquid nitrogen for 1 minute. After melting and adding lysis buffer, the lysate is homogenized by 10-20 strokes.
2. Cellular lysate from 1010 mammalian cells in 10 ml Lysis Buffer (50 mM KCl, 10 mM HEPES pH 7.4, 10% glycerol, 1% DMSO, 0.1% TritonX100, 1 mM PMSF, 1 mM benzamidine) is centrifuged at 15,000 rpm for 30 minutes and the supematant is transferred to a clean tube.
3. Adding 200 microliters of antibody coated beads and rotating the tube for 1.5 hours.
4. Centrifugation at 2,000 rpm for 3 minutes and decanting the supernatant.
5. Transferring the beads to a 10 ml chromatography column. Washing the column with 10 ml washing buffer (WB) by applying 200 microliter aliquots (WB: 50 mM KCl, 10 mM HEPES pH 7.4, 7% glycerol, 1% DMSO, 0.1% TritonX100).
6. Transferring the beads to a 5 ml tube and adding 3 ml WB containing 0.3M KCl (WB(0.3M)).
7. Rotating the tube for 10 minutes.
8. Transferring the beads and the buffer to a 10 ml chromatography column and collecting the eluate by gravity flow. Washing the beads with two column volumes of WB(0.3M) and combining the eluates.
9. Analyzing the eluate.

The previous protocol can be modified in order to avoid the immobilization of the antibody. An IgG type antibody can be raised against the protein of interest and, after binding to the corresponding protein of interest, the antibody can be immobilized on Protein A—Sepharose. After removal of the unbound substances, biomolecules that are bound to the protein of interest are separated from it by increasing the salt concentration to 0.35M KCl. The following protocol can be used (all manipulations are performed at 0° C. to 5° C):
1. The cells are collected and frozen in liquid nitrogen for 1 minute. After melting and adding lysis buffer, the lysate is homogenized by 10-20 strokes.
2. Cellular lysate from 5×109 mammalian cells in 10 ml Lysis Buffer (50 mM KCl, 10 mM HEPES pH 7.4, 10% glycerol, 1% DMSO, 0.1% TritonX100, 1 mM PMSF, 1 mM benzamidine, 0.5 mM MgCl2, 0.1 mM DTT) is centrifuged at 15,000 rpm for 20 minutes and the supernatant is transferred to a clean tube.
3. Adding the antibody and incubating for 1.5 hours.
4. Adding 300 microliters of Protein A-sepharose beads and rotating the tube for 1 hour.
5. Centrifugation at 2,000 rpm for 3 minutes and decanting the supematant.
6. Transferring the beads to a 10 ml chromatography column. Washing the column with 10 ml washing buffer (WB) by applying 200 microliter aliquots (WB—50 mM KCl, 10 mM HEPES pH 7.4, 7% glycerol, 1% DMSO, 0.1% TritonX100, 0.5 mM MgCl2, 0.1 mM DTT).
7. Transferring the beads to a 5 ml tube and adding 3 ml WB containing 0.35M KCl (WB(0.35M)).
8. Rotating the tube for 15 minutes.
9. Transferring the beads and the buffer to a 10 ml chromatography column and collecting the eluate by gravity flow. Washing the beads with two column volumes of WB (0.35M) and combining the eluates.
10. Analyzing the eluate.

Another variation consists of using biotinylated antibodies and utilizing their strong binding to streptavidin. The antibody can be biotinylated and added to the cellular lysate and, after 1 hour incubation, the lysate can be mixed with streptavidin coated sepharose beads. The following protocol can be used (all manipulations are performed at 0° C. to 5° C):
1. The cells are collected and frozen in liquid nitrogen for 1 minute. After melting and adding lysis buffer, the lysate is homogenized by 10-20 strokes.
2. Cellular lysate from 1010 mammalian cells in 10 ml Lysis Buffer (50 mM KCl, 10 mM HEPES pH 7.4, 10% glycerol, 1% DMSO, 0.1% TritonX100, 1 mM PMSF, 1 mM benzamidine, 0.5 mM MgCl2, 0.1 mM DTT) is centrifuged at 16,000 rpm for 30 minutes and the supernatant is transferred to a clean tube.
3. Adding biotinylated antibody and rotating the tube for 1.5 hours.
4. Adding 200 microliters of streptavidin-sepharose beads and rotating the tube for 1 hour.
5. Centrifugation at 3,000 rpm for 2 minutes and decanting the supernatant.
6. Transferring the beads to 10 ml chromatography column. Washing the column with 10 ml washing buffer (WB) by applying 200 microliter aliquots (WB—50 mM KCl, 10 mM HEPES pH 7.4, 5% glycerol, 1% DMSO, 0.1% TritonX100).
7. Transferring the beads to a 5 ml tube and adding 4 ml WB containing 0.3M KCl (WB(0.3M)).
8. Rotating the tube for 10 minutes.
9. Transferring the beads and the buffer to a 10 ml chromatography column and collecting the eluate by gravity flow. Washing the beads with two column volumes of WB(0.3M) and combining the eluates.
10. Analyzing the eluate.

Proteins that interact with glycoproteins can be isolated by the method of the invention. Cellular lysate or a fraction of cellular lysate or other biological fluid from an organism or cell culture without a genetically modified gene (i.e. devoid of an affinity tag) is mixed with lectin-coated beads in order to immobilize the glycoproteins. After removing the unbound substances, biomolecules that interact with glycoproteins are separated from them by increasing the salt concentration to 0.4-0.5 M KCl. In this case, what is isolated is not a group of proteins that interact with a particular glycoprotein, but proteins or other substances that interact with any glycoprotein that is immobilized on the lectin matrix. Since glycolipids and polysaccharides also bind to lectin, it is recommended to perform one purification step before the method of the invention in order to obtain a fraction enriched in glycoproteins. The following protocol can be used (all manipulations are performed at 0° C. to 5° C):
1. The cells are collected and frozen in liquid nitrogen for 1 minute. After melting and adding lysis buffer, the lysate is homogenized by 10-20 strokes.
2. Cellular lysate from 1010 mammalian cells in 10 ml Lysis Buffer (50 mM KCl, 10 mM HEPES pH 7.4, 10% glycerol, 1% DMSO, 0.1% TritonX100, 1 mM PMSF, 1 mM benzamidine, 1 mM MnCl2, 1 mM CaCl2) is centrifuged at 16,000 rpm for 30 minutes and the supernatant is transferred to a clean tube. (Optionally, the lysate can be fractionated in order to obtain a fraction that is enriched in glycoproteins or a particular glycoprotein).
3. Adding 200 microliters of lectin coated beads and rotating the tube for 1 hour.
4. Centrifugation at 3,000 rpm for 2 minutes and decanting the supernatant.
5. Transferring the lectin beads to a 10 ml chromatography column.

Washing the column with 10 ml washing buffer (WB) by applying 200 microliter aliquots (WB—50 mM KCl, 10 mM HEPES pH 7.4, 5% glycerol, 1% DMSO, 0.1% TritonX100, 1 mM MnCl₂, 1 mM CaCl₂).
6. Transferring the beads to a 5 ml tube and adding 3.5 ml WB containing 0.4M KCl (WB(0.4M)).
7. Rotating the tube for 10 minutes.
8. Transferring the beads and the buffer to a 10 ml chromatography column and collecting the eluate by gravity flow. Washing the beads with two column volumes of WB(0.4M) and combining the eluates.
9. Analyzing the eluate.

Another way to immobilize the protein of interest is by metal chelate affinity (e.g. poly-His tag) and to elute the interacting proteins by increasing the ionic strength of the medium.

In addition, the protein of interest can be immobilized by predominantly electrostatic forces (e.g. by Flag-tag) and the elution can be performed with an agent that affects the strength of hydrophobic bonds (e.g. ethylene glycol).

### Example 8

### Other Variations and Considerations of the Method of the Invention

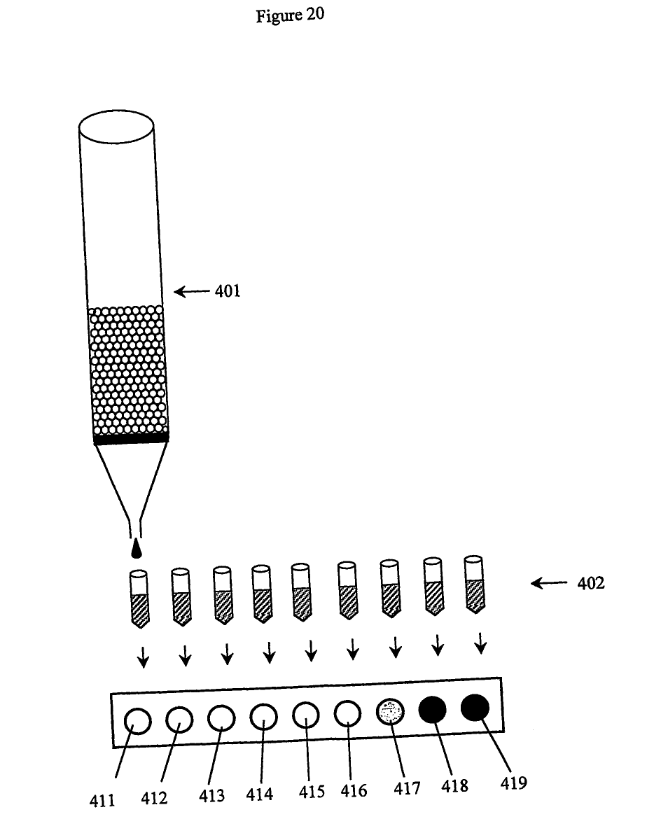

The elution agent might partially disrupt the bonding between the affinity tag and the affinity matrix. FIG. 20 illustrates elution by a gradient and collecting different fractions and analyzing each one (by immunodetection for example) for the presence of the affinity tag or the affinity tagged protein: 401—chromatography column; 402—nine subsequent fractions, small amount of each one are blotted to a membrane and analyzed; 411, 412, 413, 414, 415, 416—blots corresponding to fractions that do not contain affinity tagged protein, 417, 418, 419—blots corresponding to fractions that contain increasing amounts of the affinity tagged protein.

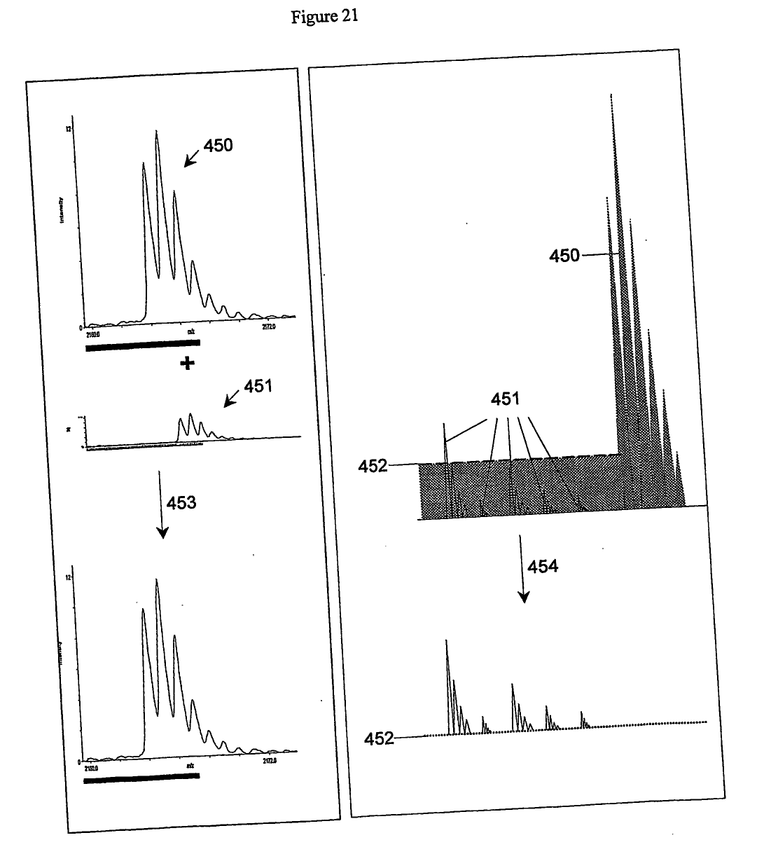

FIG. 21 illustrates the problems with analyzing fractions or gel bands containing proteins with different abundances by mass spectrometry. After the trypsin digest, the concentration range of the peptides remains approximately the same and it results in mass spectrum containing peaks with different intensity.

Left panel: When the mass spectrum contains large and small peaks separated by only a few (1-5) mass units, the detection of the latter is impossible because of the dynamic range problem in mass spectrometry. Analyzing such spectrum leads to the identification of only the major protein(s) in the band.

Right panel: High background noise problem in mass spectrometry—when a protein mixture with high dynamic range (ratio between the most abundant and the least abundant protein) is analyzed directly the small peptides are not detected because their intensities are lower than the background.

Legend: 450—high abundance peptide; 451—different low abundance peptides; 452—background of the mass spectrum; 453—low abundance peptide is not detected because it is obscured by a high abundance peptide which mass is two mass units smaller; 454—the separation of the substoichiometrically interacting proteins from the high abundance protein of interest results in lower background (due to the absence of the high abundance peptides) and makes possible the detection of the low abundance peptides.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### Full Citations for References Referred to in the Specification

Fuchs, S., Xu, S. Y., Caballero, M., Salcedo, M., La, O., Wedemann, H., and Gal, A. A missense point mutation (Leu13Arg) of the Norrie disease gene in a large Cuban kindred with Norrie disease. 1994; Hum.Mol.Genet. 3: 655-656.
Fuchs, S., Kellner, U., Wedemann, H., and Gal, A. Missense mutation C-T (Arg121Trp) in the Norrie disease gene associated with x-linked exudative vitreoretinopathy. 1995; Hum.Mutat. 6: 257-259.
Shastry, B. S., Hejtmancik, J. F., and Trese, M. T. Identification of novel missense mutations in the Norrie disease gene associated with one X-linked and four sporadic cases of familial exudative vitreoretinopathy. 1997; Hum.Mutat. 9: 396-401.
Fuentes, J. J., Volpini, V., Fernandez Toral, F., Coto, E., and Estivill, X. Identification of two new missense mutations (K58N and R121Q) in the Norrie disease (ND) gene in two Spanish families. 1993; Hum.Mol.Genet. 2: 1.953-1955.
Meindi, A., Berger, W., Meitinger, T., van de Pol, D., Achatz, H., Dorner, C., Haasemann, M., Hellebrand, H., Gal, A., and Cremers, F. Norrie disease is caused by mutations in an extracellular protein resembling C-terminal globular domain of mucins. 1992; Nat.Gen. 2:139-143.
Zaremba, J., Feil, S., Juszko, J., Myga, W., van Duijnhoven, G., and Berger, W. Intrafamilial variability of the ocular phenotype in a Polish family with a missense mutation (A63D) in the Norrie disease gene. 1998; Ophthalmic Genet. 19: 157-164.
Santschi E M, Purdy A K, Valberg S J, Vrotsos P D, Kaese H, Mickelson J R., Endothelin receptor B polymorphism associated with lethal white foal syndrome in horses. Mamm Genome 1998 Apr; 9(4): 306-9.

List of diseases caused by a point mutations that lead to disappearance or appearance of charged amino acids (some exceptions are included). Some of the mutations could be pathogenic by affecting the protein folding and/or changing the protein structure. However, some might make impossible the formation or disintegration of transient protein complexes, and/or lead to formation of unnatural permanent protein complexes (e.g. Sickle Cell Anemia).

TABLE 1
List of Diseases

| Mutation | |
|---|---|
| (Substitution) | Disease |
| E6V | Sickle Cell Anemia |
| | (formation of unnatural protein complexes) |
| | The first illness understood at the |
| | molecular level. |
| T226R | Mutation in the human voltage-gated potassium |
| | channel gene (Kv1.1) associated with: |
| | 1-episodic ataxia type 1 |
| | 2-partial epilepsy |
| D79N | Epilepsy |
| G→R | ACH—achondroplasia |
| | All persons with ACH exhibit the mutation. |
| R245X | Usher syndrome |
| | Type1 gene, PCDH15 |
| E200K | Creutzfeldt-Jakob Disease (CJD) |
| G12R | Pancreatic Adenocarcinoma - |
| G12D | Mutational in the K-ras oncogene at codon |
| | 12 has been demonstrated in 71 to 100% |
| | of the cases. |
| R506Q | Factor V Leiden of inherited thrombophilia |
| | (hypercoagulable state is caused by |
| | Activated Protein C Resistance) |
| R79C | Alexander's Disease GFAP gene |
| R79H | |
| R239C | |
| M73R R79G | |
| R88C Y242D | |
| E373X R416W | |
| H63D | Acute iron poisoning |
| H63D | Hereditary hemochromatosis (HH) |
| C282Y | |
| R−>G | Aortic Aneurysm |
| R170G | Tay-Sachs |
| | (Around 30 mutations described) |
| G145R | Mutated strain of HBV -vaccine escape |
| | mutant of hepatitis virus |
| | Mutation in the S gene |
| R519C | Mild Spondyloepiphyseal dysplasia |
| R75C | (SED) and precocious generalized |
| | osteoarthritis (OA) |
| E134K | Spinal Muscular Atrophy (SMA) - Mutation |
| | in the central Tudor-domain of the |
| | SMN protein |
| R788W | XP—xeroderma pigmentosum |
| in the XPF gene | complementation group F (XP-F) |
| K304E | Medium chain acyl CoA dehydrogenase |
| | (MCAD) Deficiency |
| R519C | Heterogeneous cartilage disorders |
| collagen II | including some chondrodysplasias and |
| | certain forms of heritable osteoarthritis |
| D816T | Systemic mast cell disease (SMCD) |
| D816V | |
| D820G | |
| W184R | Neonatal Type 2 Gaucher Disease |
| R296C | C2938T polymorphism in Cytochrome P450 |
| | CYP2D6 - responsible for the metabolism |
| | of approximately 20-25% of |
| | prescription medicines. |
| Q−>W | MED—multiple epiphyseal dysplasia, |
| COL9A2 gene | (one type of Chondrodysplasia) |
| R−>W | Collagen IX mutations |
| COL9A3 gene | |
| G1489E | EHLERS-DANLOS SYNDROME (EDS) |
| G286E | Mutations in the triple-helical |
| G661R | domain of type III procollagen |
| G1006E | (COL3A1) |
| G589E | |
| G400E | |
| G1003D | |
| G934E | |
| G499D | |
| V793G | |
| G637S | |
| G910V | |
| G595C | |
| Ile118−>Lys | Overo lethal white syndrome (OLWS) |
| (the first | is an inherited syndrome of foals born |
| transmembrane | to American Paint Horse parents of the |
| domain of | overo coat-pattern lineage. Affected |
| this seven- | foals are totally or almost totally |
| transmembrane | white and die within days from |
| domain G- | complications due to intestinal |
| protein-coupled | aganglionosis. Related conditions occur |
| receptor protein) | in humans and rodents in which mutations |
| | in the endothelin receptor B (EDNRB) |
| | gene are responsible. |

TABLE 2
Alzheimer mutations

| Mutation | Name | Description |
|---|---|---|
| E693G | ‘Arctic’ | enhanced Aβ protofibril formation |
| | APP mutation | |
| E693Q | Dutch mutation | intracerebral hemorrhages |
| A692G | Flemish mutation | Intracerebral hemorrhage. And |
| | | individuals who survive develop a |
| | | progressive dementia with features of |
| | | AD |
| E693K | Italian mutation | Clinical manifestations similar to |
| | | Dutch patients |
| D694N | Iowa mutation | Progressive dementia and severe |
| | | cerebral amyloidangiopathy |

1. A method of separating a first ligand from a second ligand in an affinity matrix system, wherein the first ligand associates in vivo with the second ligand and wherein the first ligand and the second ligand associate with each other by electrostatic forces, comprising the steps:
(a) Obtaining a sample containing biological complexes that include the first ligand and the second ligand;
(b) Immobilizing the second ligand on an affinity matrix;
(c) Removing unbound substances from the affinity matrix;
(d) Separating the first ligand from the immobilized second ligand, which remains bound to the affinity matrix during the separation, by decreasing the electrostatic forces between the first ligand and the second ligand; and
(e) Optionally, analyzing the separated first ligand.

2. A method of separating a first ligand from a second ligand in an affinity matrix system, wherein the first ligand associates in vivo with the second ligand, wherein the first ligand and the second ligand associate with each other by electrostatic forces, and wherein the second ligand is a recombinant fusion protein containing at least one affinity tag, comprising the steps:
(a) Introducing into a cell or organism a recombinant nucleic acid molecule encoding a fusion protein comprising the second ligand fused to at least one affinity tag that can selectively bind to an affinity matrix;
(b) Expressing the fusion protein;
(c) Obtaining a sample containing biological complexes that include the first ligand and the fusion protein;
(d) Immobilizing the fusion protein on the affinity matrix via the affinity tag;
(e) Removing unbound substances from the affinity matrix;
(f) Separating the first ligand from the immobilized fusion protein, which remains bound to the affinity matrix during the separation, by decreasing the electrostatic forces between the first ligand and the fusion protein; and
(g) Optionally, analyzing the separated first ligand.

3. A method of separating a first ligand from a second ligand in an affinity matrix system, wherein the first ligand associates in vivo with the second ligand, wherein the first ligand and the second ligand associate with each other by electrostatic forces, and wherein the second ligand is a recombinant fusion protein containing at least two different affinity tags, comprising the steps:
(a) Introducing into a cell or organism a recombinant nucleic acid molecule encoding a fusion protein comprising the second ligand fused to at least two different affinity tags that can selectively bind to different affinity matrixes;
(b) Expressing the fusion protein;
(c) Obtaining a sample containing biological complexes that include the first ligand and the fusion protein;
(d) Immobilizing the fusion protein on a first affinity matrix via a first affinity tag;
(e) Removing unbound substances from the first affinity matrix;
(f) Separating the fusion protein from the first affinity matrix;
(g) Immobilizing the fusion protein on a second affinity matrix via a second affinity tag, which is different than the first affinity tag;
(h) Removing unbound substances from the second affinity matrix;
(i) Separating the first ligand from the immobilized fusion protein, which remains bound to the second affinity matrix during the separation, by decreasing the electrostatic forces between the first ligand and the fusion protein; and
(j) Optionally, analyzing the separated first ligand.

4. A method of separating a first ligand from a second ligand in an affinity matrix system, wherein the first ligand associates in vivo with the second ligand, wherein the first ligand and the second ligand associate with each other by electrostatic forces, and wherein the second ligand is a protein complex containing two or more subunits of which are fused to different affinity tags, comprising the steps:
(a) Introducing into a cell or organism recombinant nucleic acids molecules encoding fusion proteins comprising the two or more subunits of which are fused to different affinity tags that can selectively bind to different affinity matrixes;
(b) Expressing the fusion proteins;
(c) Obtaining a sample containing biological complexes that include the first ligand and the fusion proteins;
(d) Immobilizing the fusion proteins on a first affinity matrix via a first affinity tag;
(e) Removing unbound substances from the first affinity matrix;
(f) Separating the fusion proteins from the first affinity matrix;
(g) Immobilizing the fusion proteins on a second affinity matrix via second affinity tag, which is different than the first affinity tag;
(h) Removing unbound substances from the second affinity matrix;
(i) Separating the first ligand from the immobilized fusion proteins, which remain bound to the second affinity matrix during the separation, by decreasing the electrostatic forces between the first ligand and the fusion proteins; and
(j) Optionally, analyzing the separated first ligand.

5. The method according to claim 1, wherein the second ligand is immobilized selectively on the affinity matrix coated with antibody that binds to the second ligand.

6. The method according to claim 2, wherein the at least one affinity tag can bind selectively to the Fc domains of immunoglobulin.

7. The method according to claim 6, wherein the at least one affinity tag contains one or more IgG binding regions of Staphylococcus aureus Protein A or Streptococcal protein G.

8. The method according to claim 2, wherein the at least one affinity tag can be separated selectively from the affinity matrix by treatment with a chemical agent.

9. The method according to claim 8, wherein the at least one affinity tag is selected from the group consisting of GST-tag, SBP-tag, calmodulin binding peptide and maltose binding peptide.

10. The method according to claim 3, wherein the second ligand is separated from the first affinity matrix by enzymatic cleavage.

11. The method according to claim 10, wherein the enzymatic cleavage is cleavage by TEV protease.

12. The method according to claim 3, wherein the immobilization in step (d) is performed by binding to a solid support coated with a specific antibody and the removal in step (e) is performed by addition of the same antibody.

13. The method according to claim 3, wherein the immobilization in step (g) is performed by binding to a solid support coated with a specific antibody.

14. The method according to claim 3, wherein the first affinity tag can bind selectively to the Fc domains of immunoglobulin.

15. The method according to claim 14, wherein the first tag contains one or more IgG binding regions of Staphylococcus aureus Protein A (SpA-tag) or Streptococcal protein G (SpG-tag).

16. The method according to claim 3, wherein the first affinity tag can be separated selectively from the first affinity matrix by treatment with a chemical agent.

17. The method according to claim 16, wherein the first affinity tag is from the group consisting of GST-tag, SBP-tag, calmodulin binding peptide and maltose binding peptide.

18. The method according to claim 3, wherein the second affinity tag can be separated from the first affinity matrix by treatment with a chemical agent.

19. The method according to claim 18, wherein, the second affinity tag is from the group consisting of GST-tag, SBP-tag, calmodulin binding peptide and maltose binding peptide.

20. The method according to claim 3, wherein the second affinity tag can bind selectively to the Fc domains of immunoglobulin.

21. The method according to claim 20, wherein the second tag contains one or more IgG binding regions of Staphylococcus aureus Protein A (SpA-tag) or Streptococcal protein G (SpG-tag).

22. The method according to claim 1, wherein the first and second ligand are separated by decreasing the electrostatic forces between the first and second ligand by increasing the ionic strength of the system.

23. The method according to claim 22, wherein the ionic strength of the system is increased with a chemical agent.

24. The method according to claim 23, wherein the chemical agent is KCl.

25. The method according to claim 23, wherein the change of the concentration of the chemical agent is less than 30 mM.

26. The method according to claim 23, wherein the change of the concentration of the chemical agent is between 30 mM and 2M.

27. (canceled)

28. (canceled)

29. The method according to claim 1, wherein the first and second ligand are separated by decreasing the electrostatic forces between the first and second ligand by changing the pH of the system.

30. The method according to claim 1, wherein the first ligand is separated from the second ligand by enzymatic treatment that modifies the second ligand.

31. The method according to claim 1, wherein the first ligand is separated from the second ligand by enzymatic treatment that modifies the first ligand.

32. The method according claim 1, wherein after the separation of the second ligand from the first ligand, the immobilized second ligand is mixed with a cellular lysate from a different or same organism, and after removal of the unbound substances, the first ligand is separated from the second ligand.

33. The method according to claim 32, wherein an affinity tagged second ligand is covalently cross-linked to the affinity matrix after the separation of the second ligand from the first ligand and before the immobilized second ligand is mixed with a cellular lysate from a different or same organism.

34. The method according to claim 1, wherein a chemical or biomolecule is identified as a drug or pre-drug by its capability to separate selectively the first ligand from the second ligand and/or affect selectively the separation of the first ligand from the second ligand when it is added to or removed from the cellular lysate and/or the affinity matrix system and wherein the change of the concentration of the chemical or biomolecule is below 30 mM.

35. The method according to claim 34, wherein the second ligand is a protein, which contains at least one mutation.

36. The method according to claim 34, wherein the second ligand associates directly or indirectly with a protein that contains at least one mutation.

37. The method according to claims 34, wherein the chemical or biomolecule is designed, synthesized and/or selected for testing by the capability of the chemical or biomolecule to bind to the first ligand or second ligand.

38. The method according to claim 34, wherein the chemical or biomolecule is designed, synthesized and/or selected for testing by one or more of the following features: (a) capability of the chemical or biomolecule to bind selectively to the mutated protein; (b) the chemical or biomolecule contains at least one electrostatic charge that is identical to the charge that has been changed as a result of the mutation, and (c) after binding of the chemical or biomolecule to the mutated protein, the electrostatic charge is located at distance between 0 and 0.5 nanometers from the mutated amino acid in the mutated protein.

39. The method according to claim 1, wherein the second ligand is an enzyme.

40. The method according to claim 1, wherein the second ligand is a substrate for an enzyme.

41. The method according to claim 1, wherein the second ligand is an enzyme and a substrate for a different enzyme.

42. The method according to claim 41, wherein the second ligand is RNA polymerase or DNA polymerase.

43. A method for identifying protein-protein association as a putative cause for a disease, comprising associating the disease with a mutation that changes the electrostatic properties of a protein by replacing Lysine and/or Arginine and/or Aspartate and/or Glutamate and/or Histidine with an uncharged or oppositely charged amino acid.

44. A method for identifying protein-protein association as a putative cause for a disease, comprising associating the disease with a mutation that changes the electrostatic properties of a protein by replacing an amino acid with Lysine and/or Arginine and/or Aspartate and/or Glutamate and/or Histidine.

45. The method according to claim 1, wherein protein-protein association as a putative cause for a disease is identified.

46. The method of claim 45, wherein the disease comprises Norrie disease, Alzheimer's disease, Parkinson's disease, beta3-adrenergic receptor gene mutation, achondroplasia, sickle cell anemia, thrombosis, epilepsy, Usher syndrome, Creutzfeldt-Jakob disease, Dancreatic adenocarcinoma, Alexander's disease, aortic aneurysm, Tay-Sachs disease, xeroderma piamentosum, systemic mast cell disease, neonatal type 2 Gaucher disease, multiple epiphyseal dysplasia, Ehlers-Danlos syndrome, or alpha 1-antitrypsin deficiency.

47. The method according to claim 1, wherein the second ligand is a glycoprotein and the affinity matrix is lectin coated beads.

48. The method according to claim 1, wherein the second ligand is a nucleic acid, which is part of a nucleoprotein complex and the affinity matrix consists of immobilized nucleic acid with complementary sequence.

49. The method according to claim 1, wherein the second ligand is a nucleic acid, which is part of a nucleoprotein complex and is genetically engineered so that it contains a poly-Guanosine and the affinity matrix consists of immobilized poly-dCytosine.

50. A reagent kit comprising a buffer for preparation of cellular lysate and washing buffer and wherein the buffers are capable of maintaining the electrostatic forces between the first ligand and the second ligand.

51. The reagent kit according to claim 50 wherein the ionic strength of the buffers is between 0 and 400 mM.

52. The reagent kit according to claim 50 additionally comprising at least one chemical agent for separating the first ligand from the second ligand and wherein the chemical agent is capable of decreasing the electrostatic forces between the first ligand and the second ligand.
